(19) United States
(12) Patent Application Publication
(10) Pub. No.: US 2022/0073934 A1
(43) Pub. Date: Mar. 10, 2022

(54) Beta-Galactosidase Alpha Peptide as a Non-Antibiotic Selection Marker and Uses Thereof

(71) Applicant: Janssen Biotech, Inc., Horsham, PA (US)

(72) Inventor: William Perry, Warrington, PA (US)

(73) Assignee: Janssen Biotech, Inc., Horsham, PA (US)

(21) Appl. No.: 17/417,022

(22) PCT Filed: Jan. 14, 2020
(86) PCT No.: PCT/IB2020/050267

§ 371 Date: Jun. 21, 2021

Related U.S. Application Data
(60) Provisional application No. 62/793,933, filed on Jan. 18, 2019.

(51) Int. Cl. C12N 15/72; C12N 15/65; C12N 15/81; C12N 9/38

(52) U.S. Cl. CPC C12N 15/72; C12N 15/65; C12N 15/81; C12Y 302/01023; C12N 2800/101; C12N 2800/102; C12N 2820/55; C12N 9/2471

(57) ABSTRACT

Provided herein are methods of using a nucleic acid construct as a selectable marker. The nucleic acid construct comprises an isolated β-galactosidase expression cassette comprising a nucleic acid sequence encoding the amino-terminal fragment of β-galactosidase operably linked to a promoter. Also provided are isolated vectors comprising the β-galactosidase expression cassette, methods of generating the isolated vector, and kits comprising the isolated vector.

### FIELD OF THE INVENTION

This invention relates to isolated β-galactosidase expression cassettes comprising a non-antibiotic selection marker. Specifically, the isolated β-galactosidase expression cassettes comprise the amino-terminal fragment of β-galactosidase operably linked to a promoter. Also provided are isolated vectors comprising the β-galactosidase expression cassettes, methods of producing the isolated vectors, and kits comprising the isolated vectors.

### REFERENCE TO SEQUENCE LISTING SUBMITTED ELECTRONICALLY

This application contains a sequence listing, which is submitted electronically via EFS-Web as an ASCII formatted sequence listing with a file name “JBI6031USPSP1Seqlist1” and a creation date of Jan. 17, 2019 and having a size of 48 kb. The sequence listing submitted via EFS-Web is part of the specification and is herein incorporated by reference in its entirety.

### BACKGROUND OF THE INVENTION

Plasmid vectors usually contain genes that are expressed in E. coli and provide a way to identify or select cells containing the plasmid from those which do not contain the plasmid when the plasmid is introduced into cells by transformation or electroporation. The most commonly used selectable markers are genes that confer resistance to antibiotics. However, there are several situations where antibiotic resistance genes are undesirable. When plasmids are used to create manufacturing cell lines for biologics such as antibodies, the antibiotic resistance genes are usually removed or destroyed. For gene therapies, antibiotic resistance genes are also undesirable. While the kanamycin/neomycin resistance gene is often tolerated by the FDA, EU regulatory agencies are much stricter. The European Pharmacopei states “Unless otherwise justified and authorized, antibiotic resistance genes used as selectable genetic markers, particularly for clinically useful antibiotics, are not included in the vector construct. Other selection techniques for the recombinant plasmid are preferred” (“Gene transfer medical products for human use.” European Pharmacopei 7.0 (2011)). While destruction of the antibiotic selection marker may be possible when a small amount of the plasmid is needed for cell line development, these techniques are impractical for gene therapy applications where more of the plasmid needs to be manufactured.

Plasmid vectors where the replication origin and selection marker are a combined size of <1 kb are needed for development of plasmid-based gene therapies to avoid gene silencing in vivo. Therapeutic transgenes were expressed longer and at higher levels in mice when the plasmid backbones were 1 kb or less compared to traditional plasmids with plasmid backbones 3 kb or more (Lu et al., Mol. Ther. 20(11):2111-9 (2012)). It was proposed that large blocks of DNA that were not expressed in vivo induced silencing. Thus, plasmids with smaller plasmid backbones might be much more efficacious.

Smaller plasmids are also needed for applications where transient transfection is used to manufacture therapeutics. One example is the production of Adeno-associated viral vectors where large-scale transfection of plasmids is used to generate clinical material. Smaller plasmids reduce the amount of DNA that must be transfected, reducing costs.

Thus, there is a need for generating smaller plasmids comprising a selectable marker that can be used for gene therapy applications.

### BRIEF SUMMARY OF THE INVENTION

In one general aspect, provided are methods of using a nucleic acid construct as a selectable marker. The methods comprise (a) contacting a host cell comprising a deletion in a lac operon with the nucleic acid construct, wherein the nucleic acid construct comprises an isolated β-galactosidase expression cassette comprising a nucleic acid sequence encoding the amino-terminal fragment of β-galactosidase operably linked to a promoter; and (b) growing the host cell under conditions wherein the nucleic acid construct is maintained in the host cell.

In another general aspect, provided are isolated β-galactosidase expression cassettes. The isolated cassette comprises a nucleic acid sequence encoding the amino-terminal fragment of β-galactosidase operably linked to a promoter.

In certain embodiments, the amino-terminal fragment of β-galactosidase comprises an amino acid sequence with at least 75% identity to SEQ ID NO:1. In certain embodiments, the amino-terminal fragment of β-galactosidase comprises an amino acid sequence of SEQ ID NO:1.

In certain embodiments, the nucleic acid sequence further comprises a replication origin. The replication origin can, for example, be a high-copy replication origin. In certain embodiments, the high-copy replication origin is the pUC57 replication origin. In certain embodiments, the pUC57 replication origin comprises the nucleic acid sequence of SEQ ID NO:19.

In certain embodiments, the isolated β-galactosidase expression cassette further comprises a dimer resolution element. The dimer resolution element can, for example, comprise a nucleic acid sequence comprising a site-specific recombinase recognition site. The dimer resolution element can further comprise a nucleic acid sequence encoding a site specific recombinase. In certain embodiments, the host cell comprises a nucleic acid sequence encoding a site-specific recombinase. The dimer resolution element can, for example, be a ColE1 dimer resolution element. In certain embodiments, the ColE1 dimer resolution element comprises the nucleic acid sequence of SEQ ID NO:20.

Also provided are isolated vectors comprising the isolated β-galactosidase expression cassettes of the invention. In certain embodiments, the isolated vector is less than about 1.5 kilobases in size. In certain embodiments, the isolated vector comprises a nucleic acid sequence selected from the group consisting of SEQ ID NOs:9-13, 17, and 18.

Also provided are methods of generating the isolated vectors of the invention. The methods comprise (a) contacting a host cell with the isolated vector; (b) growing the host cell under conditions to produce the vector; and (c) isolating the vector from the host cell.

In certain embodiments, the host cell is grown in minimal media. The minimal media can comprise lactose as the sole carbon source. In certain embodiments, the minimal media comprises about 1% to about 4% weight per volume (w/v) lactose. In certain embodiments, the minimal media comprises about 2% w/v lactose.

Also provided are kits comprising (a) an isolated β-galactosidase expression cassette of the invention; and (b) a host cell comprising a deletion in a lac operon. In certain embodiments, the kit further comprises minimal media comprising lactose as the sole carbon source. In certain embodiments, a vector comprises the isolated β-galactosidase expression cassette. In certain embodiments, the host cell comprises the LacZΔM15 deletion. In certain embodiments, the host cell is selected from the group consisting of an E. coli host cell and a yeast host cell.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a schematic of the P215 plasmid.

FIG. 2 shows a schematic of the P216 plasmid.

FIG. 3 shows a schematic of the P217 plasmid.

FIG. 4 shows a schematic of the P218 plasmid.

FIG. 5 shows a schematic of the P219 plasmid.

FIG. 6 shows a schematic of the P469-2 plasmid.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., amino-terminal β-gacatosidase peptides and polynucleotides that encode them; nucleic acids of the isolated vectors described herein), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture, or anchored to the cell membrane.

The term “operatively linked” as used herein, refers to the linkage between nucleic acids (e.g., a promoter and a nucleic acid encoding a polypeptide) when it is placed into a structural or functional relationship. For example, one segment of a nucleic acid sequence can be operably linked to another segment of a nucleic acid sequence if they are positioned relative to one another on the same contiguous nucleic acid sequence and have a structural or functional relationship, such as a promoter or enhancer that is positioned relative to a coding sequence so as to facilitate transcription of the coding sequence; a ribosome binding site that is positioned relative to a coding sequence so as to facilitate translation; or a pre-sequence or secretory leader that is positioned relative to a coding sequence so as to facilitate expression of a pre-protein (e.g., a pre-protein that participates in the secretion of the encoded polypeptide). In other examples, the operably linked nucleic acid sequences are not contiguous, but are positioned in such a way that they have a functional relationship with each other as nucleic acids or as proteins that are expressed by them. Enhancers, for example, do not have to be contiguous. Linking can be accomplished by ligation at convenient restrictions sites or by using synthetic oligonucleotide adaptors or linkers.

The term “promoter” as used herein, refers to a nucleic acid sequence enabling the initiation of the transcription of a gene sequence in a messenger RNA, such transcription being initiated with the binding of an RNA polymerase on or nearby the promoter.

The term “replication origin” or “origin of replication” as used herein, refers to a nucleic acid sequence that is necessary for replication of a plasmid. Examples of replication origins include, but are not limited to, the pBR322 replication origin, the ColE1 replication origin, the pUC57 replication origin, a pMB1 replication origin, a pSC101 replication origin, and a R6K gamma replication origin. Replication origins can be high-or low-copy. A high-copy replication origin, when present in a vector, can result in a high number (e.g., 150 to 200) of copies of the vector per cell. A medium-copy replication origin, when present in a vector, can result in a medium number (e.g., 25 to 50) of copies of the vector per cell. A low-copy replication origin, when present in a vector, can result in a low number (e.g., 1 to 3) of copies of the vector per cell.

The term “dimer resolution element” as used herein, refers to a nucleic acid sequence that facilitates the in vivo conversion of multimers of the nucleic acid sequence (e.g., a vector or plasmid) to monomers in which said sequence is present. A dimer resolution element can comprise a nucleic acid sequence comprising a site-specific recombinase target site (e.g., a LoxP target site, a rfs target site, a FRT target site, a RP4 res target site, a RK2 res target site, and a res target site). A dimer resolution element can comprise a nucleic acid sequence encoding a site-specific recombinase (e.g., a Cre recombinase, a ResD recombinase, a Flp recombinase, a ParA recombinase, a Sin recombinase, a β recombinase, a γδ recombinase, a tnpR recombinase, and a pSK41 resolvase). Dimers of isolated vectors/nucleic acids can be resolved by an enzyme acting on the target DNA sequence comprised within the dimer resolution element. The enzyme recombines the target DNA sequence. By way of a non-limiting example, the enzymes XerC and XerD, expressed either by the host cell or the vector comprising the dimer resolution element, recognize the cer target site of the ColE1 dimer resolution element and work with several additional cofactors to ensure that a monomer of the vector/nucleic acid is produced.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

Polynucleotides, Vectors, Host Cells, and Methods of Use

In one general aspect, provided are methods of using a nucleic acid construct as a selectable marker. The methods comprise (a) contacting a host cell comprising a deletion in a lac operon with the nucleic acid construct, wherein the nucleic acid construct comprises an isolated β-galactosidase expression cassette comprising a nucleic acid sequence encoding the amino-terminal fragment of β-galactosidase operably linked to a promoter; and (b) growing the host cell under conditions wherein the nucleic acid construct is maintained in the host cell.

In another general aspect, the invention relates to an isolated β-galactosidase expression cassette comprising a nucleic acid sequence encoding the amino-terminal fragment of β-galactosidase operably linked to a promoter.

In certain embodiments, the amino-terminal fragment of β-galactosidase comprises an amino acid sequence with at least 75% identity to SEQ ID NO: 1. In certain embodiments, the amino-terminal fragment of β-galactosidase comprises an amino acid sequence with at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to SEQ ID NO:1. The amino-terminal fragment of the β-galactosidase can comprise SEQ ID NO:1.

In certain embodiments, the nucleic acid sequence further comprises a replication origin. The replication origin can, for example, be a high-copy replication origin. In certain embodiments, the high-copy replication origin is the pUC57 replication origin. In certain embodiments, the pUC57 replication origin comprises a nucleic acid sequence with at least 90%, 91%, 92%, 93%, 94%, 95%, 96%, 97%, 98%, or 99% identity to SEQ ID NO:19. In certain embodiments, the pUC57 replication origin comprises a nucleic acid sequence of SEQ ID NO:19.

In certain embodiments, the isolated β-galactosidase expression cassette can further comprise a dimer resolution element. The dimer resolution element can, for example, comprise a nucleic acid sequence comprising a site-specific recombinase recognition site. The site-specific recombinase recognition site can, for example, be selected from the group consisting of a LoxP site, a rfs site, a FRT site, a RP4 res site, a RK2 res site, and a res site. The dimer resolution element can further comprise a nucleic acid sequence encoding a site specific recombinase. In certain embodiments, the host cell comprises a nucleic acid sequence encoding a site-specific recombinase. The site-specific recombinase can, for example, be selected from the group consisting of a Cre recombinase, a ResD recombinase, a Flp recombinase, a ParA recombinase, a Sin recombinase, a (3 recombinase, a γδ recombinase, a tnpR recombinase, and a pSK41 resolvase.

The dimer resolution element can, for example, be a ColE1 dimer resolution element. The ColE1 dimer resolution element can comprise a nucleic acid sequence with at least 90%, 91%, 92%, 93%, 94%, 95%, 96%, 97%, 98%, or 99% identity to SEQ ID NO:20. In certain embodiments, the ColE1 dimer resolution element comprises a nucleic acid sequence of SEQ ID NO:20.

In certain embodiments, an isolated vector comprises the isolated β-galactosidase expression cassettes of the invention. Any vector known to those skilled in the art in view of the present disclosure can be used, such as a plasmid, a cosmid, an artificial chromosome (e.g., a bacterial artificial chromosome (BAC), a yeast artificial chromosome (YAC), and/or a P1-derived artificial chromosome (PAC)), a transposon, a phage vector, or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for the production of the amino-terminal fragment of the β-galactosidase peptide. Conventional cloning techniques or artificial gene synthesis can be used to generate a recombinant expression vector according to embodiments of the invention.

In certain aspects, the isolated vector is less than about 1.5 kilobases in size. The isolated vector can, for example, be about 700 base pairs, about 800 base pairs, about 900 base pairs, about 1000 base pairs (about 1 kilobase), about 1100 base pairs (about 1.1 kilobases), about 1200 base pairs (about 1.2 kilobases), about 1300 base pairs (about 1.3 kilobases), about 1400 base pairs (about 1.4 kilobases), or about 1500 base pairs (about 1.5 kilobases) in length. In certain embodiments, the isolated vector is less than about 1 kilobase in size. In certain embodiments, the isolated vector is less than about 900 base pairs in size. In certain embodiments, the isolated vector is less than about 800 base pairs in size.

In certain embodiments, the isolated vector comprises a nucleic acid sequence with at least 90%, 91%, 92%, 93%, 94%, 95%, 96%, 97%, 98%, or 99% identity to a nucleic acid selected from the group consisting of SEQ ID NOs:9-13, 17, and 18. In certain embodiments, the isolated vector comprises a nucleic acid sequence selected from the group consisting of SEQ ID NOs:9-13, 17, and 18.

Also provided are methods of generating the isolated vector of the invention. The methods comprise (a) contacting a host cell with the isolated vector; (b) growing the host cell under conditions to produce the vector; and (c) isolating the vector from the host cell.

In certain embodiments, the host cell is grown in minimal media. The minimal media can comprise lactose as the sole carbon source. In certain embodiments, the minimal media comprises about 1% to about 4% weight per volume (w/v) lactose. In certain embodiments, the minimal media comprises about 1% to about 4% w/v, about 1% to about 3% w/v, about 1% to about 2% w/v, about 1.5% to about 4% w/v, about 1.5% to about 3% w/v, about 1.5% to about 2% w/v, about 2% to about 4% w/v, about 2% to about 3% w/v, about 2.5% to about 4% w/v, about 2.5% to about 35% w/v, or about 3% to about 4% w/v lactose. In certain embodiments, the minimal media comprises about 2% w/v lactose.

In certain embodiments, the invention relates to a host cell comprising an isolated vector of the invention. Any host cell known to those skilled in the art in view of the present disclosure can be used for comprising an isolated vector of the invention. Suitable host cells include cells with the LacZΔM15 deletion but with the rest of the lactose biosynthetic pathway intact. Strains that contain this mutation in the context of the bacteriophage Φ80 integration (i.e., Φ80lacZΔM15 marker) contain this mutation in the context of the complete lac operon, and, therefore, are suitable hosts. Other hosts with different deletions in the amino-terminal (N-terminal) region of the LacZ gene, which produce significant levels of β-galactosidase when transformed with a LacZ-α complementation plasmid can also be suitable hosts. Suitable host cells of the invention can include an E. coli host cell or a yeast host cell.

Also provided are kits comprising (a) an isolated β-galactosidase expression cassette of the invention; and (b) a host cell comprising a deletion in a lac operon. In certain embodiments, a vector comprises the isolated β-galactosidase expression cassette. In certain embodiments, the host cell comprises the LacZΔM15 deletion. In certain embodiments, the host cell can be selected from an E. coli host cell or a yeast host cell.

In certain embodiments, the kit further comprises minimal media comprising lactose as the sole carbon source. In certain embodiments, the minimal media comprises about 1% to about 4% weight per volume (w/v) lactose. In certain embodiments, the minimal media comprises about 1% to about 4% w/v, about 1% to about 3% w/v, about 1% to about 2% w/v, about 1.5% to about 4% w/v, about 1.5% to about 3% w/v, about 1.5% to about 2% w/v, about 2% to about 4% w/v, about 2% to about 3% w/v, about 2.5% to about 4% w/v, about 2.5% to about 35% w/v, or about 3% to about 4% w/v lactose. In certain embodiments, the minimal media comprises about 2% w/v lactose.

### Embodiments

This invention provides the following non-limiting embodiments.

Embodiment 1 is a method of using a nucleic acid construct as a selectable marker, the method comprising:
a. contacting a host cell comprising a deletion in a lac operon with the nucleic acid construct, wherein the nucleic acid construct comprises an isolated β-galactosidase expression cassette comprising a nucleic acid sequence encoding the amino-terminal fragment of β-galactosidase operably linked to a promoter; and
b. growing the host cell under conditions wherein only the host cell containing the nucleic acid construct is maintained in the host cell.

Embodiment 2 is the method of embodiment 1, wherein the amino-terminal fragment of β-galactosidase comprises an amino acid sequence with at least 75% identity to SEQ ID NO:1.

Embodiment 3 is the method of embodiment 1 or 2, wherein the amino-terminal fragment of β-galactosidase comprises an amino acid sequence of SEQ ID NO:1.

Embodiment 4 is the method of any one of embodiments 1-3, wherein the nucleic acid sequence further comprises a replication origin.

Embodiment 5 is the method of embodiment 4, wherein the replication origin is a high-copy replication origin.

Embodiment 6 is the method of embodiment 5, wherein the high-copy replication origin is the pUC57 replication origin.

Embodiment 7 is the method of embodiment 6, wherein the pUC57 replication origin comprises the nucleic acid sequence of SEQ ID NO:19.

Embodiment 8 is the method of any one of embodiments 1-7, wherein the isolated β-galactosidase expression cassette further comprises a dimer resolution element.

Embodiment 9 is the method of embodiment 8, wherein the dimer resolution element comprises a nucleic acid sequence comprising a site-specific recombinase recognition site.

Embodiment 10 is the method of embodiment 8 or 9, wherein the dimer resolution element further comprises a nucleic acid sequence encoding a site-specific recombinase.

Embodiment 11 is the method of embodiment 8 or 9, wherein the host cell comprises a nucleic acid sequence encoding a site-specific recombinase.

Embodiment 12 is the method of any one of embodiments 8-11, wherein the dimer resolution element is a ColE1 dimer resolution element.

Embodiment 13 is the method of embodiment 12, wherein the ColE1 dimer resolution element comprises the nucleic acid sequence of SEQ ID NO:20.

Embodiment 14 is the method of any one of embodiments 1-13, wherein the host cell comprises a LacZΔM115 deletion.

Embodiment 15 is the method of any one of embodiments 1-14, wherein an isolated vector comprises the isolated β-galactosidase expression cassette.

Embodiment 16 is the method of embodiment 15, wherein the isolated vector is less than about 1.5 kilobases in size.

Embodiment 17 is the method of embodiment 15 or 16, wherein the isolated vector comprises a nucleic acid sequence selected from the group consisting of SEQ ID NOs:9-13, 17, and 18.

Embodiment 18 is a method of generating the isolated vector of any one of embodiments 15-17, wherein the method comprises:

a. contacting a host cell with the isolated vector;

b. growing the host cell under conditions to produce the vector;

c. isolating the vector from the host cell.

Embodiment 19 is the method of embodiment 18, wherein the host cell is grown in minimal media.

Embodiment 20 is the method of embodiment 19, wherein the minimal media comprises lactose as the sole carbon source.

Embodiment 21 is the method of embodiment 20, wherein the minimal media comprises about 1% to about 4% weight per volume (w/v) lactose.

Embodiment 22 is the method of embodiment 21, wherein the minimal media comprises about 2% w/v lactose.

Embodiment 23 is an isolated β-galactosidase expression cassette comprising a nucleic acid sequence encoding the amino-terminal fragment of β-galactosidase operably linked to a promoter.

Embodiment 24 is the isolated β-galactosidase expression cassette of embodiment 23, wherein the amino-terminal fragment of β-galactosidase comprises an amino acid sequence with at least 75% identity to SEQ ID NO:1.

Embodiment 25 is the isolated β-galactosidase expression cassette of embodiment 23 or 24, wherein the amino-terminal fragment of β-galactosidase comprises an amino acid sequence of SEQ ID NO:1.

Embodiment 26 is the isolated β-galactosidase expression cassette of any one of embodiments 23-25, wherein the nucleic acid sequence further comprises a replication origin.

Embodiment 27 is the isolated β-galactosidase expression cassette of embodiment 26, wherein the replication origin is a high-copy replication origin.

Embodiment 28 is the isolated β-galactosidase expression cassette of embodiment 27, wherein the high-copy replication origin is the pUC57 replication origin.

Embodiment 29 is the isolated β-galactosidase expression cassette of embodiment 28, wherein the pUC57 replication origin comprises the nucleic acid sequence of SEQ ID NO:19.

Embodiment 30 is the isolated β-galactosidase expression cassette of any one of embodiments 23-29, wherein the isolated β-galactosidase expression cassette further comprises a dimer resolution element.

Embodiment 31 is the isolated β-galactosidase expression cassette of embodiment 30, wherein the dimer resolution element comprises a nucleic acid sequence comprising a site-specific recombinase recognition site.

Embodiment 32 is the isolated β-galactosidase expression cassette of embodiment 30 or 31, wherein the dimer resolution element further comprises a nucleic acid sequence encoding a site-specific recombinase.

Embodiment 33 is the isolated β-galactosidase expression cassette of any one of embodiments 30-32, wherein the dimer resolution element is a ColE1 dimer resolution element.

Embodiment 34 is the isolated β-galactosidase expression cassette of embodiment 33, wherein the ColE1 dimer resolution element comprises the nucleic acid sequence of SEQ ID NO:20.

Embodiment 35 is an isolated vector comprising the isolated β-galactosidase expression cassette of any one of embodiments 23-34.

Embodiment 36 is the isolated vector of embodiment 35, wherein the isolated vector is less than about 1.5 kilobases in size.

Embodiment 37 is the isolated vector of embodiment 35 or 36, wherein the isolated vector comprises a nucleic acid sequence selected from the group consisting of SEQ ID NOs:9-13, 17, and 18.

Embodiment 38 is a kit comprising:
a. an isolated β-galactosidase expression cassette of any one of embodiments 23-37; and
b. a host cell comprising a deletion in a lac operon.

Embodiment 39 is the kit of embodiment 38, further comprising minimal media comprising lactose as the sole carbon source.

Embodiment 40 is the kit of embodiment 38 or 39, wherein a vector comprises the isolated β-galactosidase expression cassette.

Embodiment 41 is the kit of any one of embodiments 38-40, wherein the host cell comprises the LacZΔM15 deletion.

Embodiment 42 is the kit of embodiment 41, wherein the host cell is selected from the group consisting of an E. coli host cell and a yeast host cell.

### EXAMPLES

### Example 1: Plasmid Selection Via Alpha-Complementation of β-Galactosidase Instead of Antibiotic Selection in TOP10 Cells

### Materials

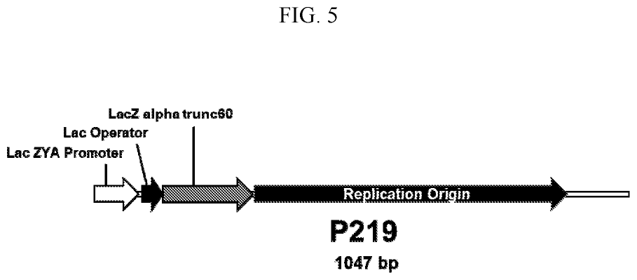

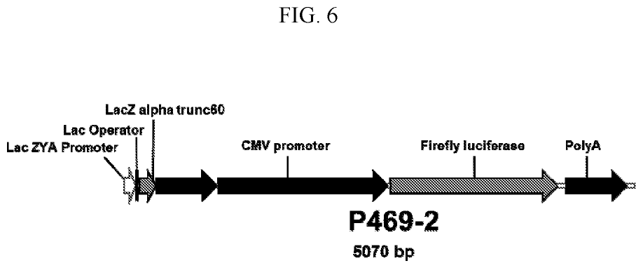

Cells: One Shot Top10 competent cells (Thermo-Fisher; Waltham, Mass., Catalog Number C404003). NEB 5-alpha (New England Biolabs, Ipswich, Mass., Catalog Number (C2987). GT115 (InvivoGen, San Diego, Calif., Catalog Number GT115-21). NEB Stable (New England Biolabs, Catalog Number C3040H). Stellar (Takara Bio USA, Mountain View, Calif., Catalog Number 636766). DH10B (Thermo-Fisher, Catalog Number 18297010). Stbl3 (Thermo-Fisher, Catalog Number C737303). Xli-blue (Agilent, Santa Clara, Calif.; Catalog Number 200236).
Plasmids: pUC19 (Thermo-Fisher Scientific; Catalog Number SD0061); pBluescript II. KS(−) (Agilent; Santa Clara, Calif.; Catalog Number 212208). Clones P215 (SEQ ID NO:9) and P216 (SEQ ID NO:10). GWIZ-Luciferase (Genlantis Corporation; San Diego, Calif.; P030200); P219 (SEQ ID NO:13; FIG. 5). P469-2 (SEQ ID NO:17; FIG. 6).
Media: M9+Lactose Media (Teknova, Hollister CA; Catalog Number M1348-04 (plates)): 0.3% KH₂PO₄, 0.6% Na₂HPO₄, 0.5% (85 mM) NaCl, 0.1% NH₄Cl, 2 mM MgSO₄, 50 mg/liter L-leucine, 50 mg/L isoleucine; 1 mM thiamine, 2% lactose, and 1.5% agar. M9+Glucose Media (Teknova Hollister CA; Catalog Number M1346-04 (plates)): 0.3% KH₂PO₄, 0.6% Na₂HPO₄, 0.5% (85 mM) NaCl, 0.1% NH₄Cl, 2 mM MgSO₄, 50 mg/liter L-leucine, 50 mg/liter isoleucine, 1 mM thiamine, 1% glucose, and 1.5% agar.
LB-Carbenicillin(100) plates (Teknova, Hollister CA; Catalog number L1010). LB Plates (Teknova Hollister CA L1100). LB+60 μg/mL X-Gal, 0.1 mM IPTG (Teknova Hollister CA L1920). SOC Media (Thermo-Fisher 15544034). LB Broth (Thermo-Fisher 10855021);
D-PBS, pH 7.1, no Mg²⁺noCa²⁺ (ThermoFisher 14200-075)

### Results

Plasmids without antibiotic selection markers are desirable for gene therapy applications and cell line development for therapeutic products. It has also been reported that plasmid backbones 1 kb or smaller were useful in avoiding gene silencing when delivered to animals in vivo. The purpose of these experiments was to explore a new strategy for developing a small metabolic selection marker for selection of plasmid-containing cells in E. coli.

It was hypothesized that plasmids that express the alpha peptide of β-galactosidase could complement the LacZΔ15 allele in TOP10 cells, completing the lactose operon and allowing cells to grow on minimal media with lactose as the sole carbon source. Plasmids pUC19 and pBluescript II both express β-galactosidase alpha peptide fusion proteins. Whether these plasmids were able to complement lac mutations in the Top10 host strain and allow growth on minimal media was tested.

To test whether pUC19 and/or pBluescript II were capable of complementing the LacZΔ15 mutations in TOP10 cells, these plasmids were transformed into the cells using the following procedure.

Two transformation mixtures were prepared in sterile microfuge tubes as follows: 1) 1 μl (100 pg) pBluescript II plasmid+50 μl One Shot TOP10 cells; 2) 1 μl (10 pg) pUC19 plasmid+50 μl One Shot TOP10 cells. The transformation mixtures were incubated on ice 30 minutes, then heat shocked for 30 seconds at 42° C. After the heat shock, the transformation mixtures were incubated on ice for 1 minute. To the transformation mixtures, 450 μl SOC media was added, and the cells were incubated shaking at 37° C. for 1 hour. The transformation mixtures containing the cells were centrifuged, and the cells were resuspended in 500 μl Sterile D-PBS buffer. The cells were centrifuged and resuspended twice more. Two 1:10 serial dilutions of the cells were made in D-PBS for each sample. 200 μl of each dilution was spread onto M9+Lactose plates. 200 μl of the first two dilutions were also spread onto LB-Carbenicillin (100) plates. The plates were incubated at 37° C. overnight.

After overnight incubation there were many colonies from both transformations plated onto LB-Carbenicillin (100) plates; these plates were stored at 4° C. There were no visible colonies from either transformation plated onto M9+Lactose plates; these plates were incubated for an additional 24 hours at 37° C. No colonies were visible on the M9-Lactose plates. Cells were cultured for an additional 48 hours at 30° C. No colonies were visible on these plates, even after extended incubation.

Neither of the cloning vectors expressing LacZ-α fusion peptides were able to complement the Lac mutation in the TOP10 host strain to allow growth in minimal media containing lactose as the sole carbon source.

It was possible that the expression of LacZ-α peptide fusion proteins by the pUC19 and pBluescript II cloning vectors was not high enough to adequately complement the lac mutations in the host strains tested. Both vectors produce fusion proteins that transcribe through the multi-cloning region and such fusion proteins could be sub-optimal for complementing the LacZΔ15 mutation.

### Example 2: LacZ Expressing Plasmids Used as a Metabolic Selection Marker in E. coli

Two LacZ-alpha expression cassettes with medium and strong promoters (LacZYA and OmpF, respectively) were designed. The OmpF promoter sequence was based on the OmpF promoter used by Stavropoulos et al. (Stavropoulos and Strathdee, Genomics 72(1):99-104 (2001)). The LacZYA promoter was derived from the sequence in pBluescript along with the lac operator sequence bound by the lac repressor.

For the open reading frame (ORF) of the LacZ alpha peptide, Reddy (Reddy, Biotechniques 37(6):948-52 (2004) reported that the plasmid pUC19 produced about 10x more beta-galactosidase activity than pBluescript. These plasmids have the same promoter elements driving the lacZ alpha peptide. However, pBluescript has a much longer polylinker than pUC19 and pUC19 encodes non-lacZ C-terminal residues. It is unknown which of these differences result in higher pUC19 beta-galactosidase activity. Nishiyama et al found that the N-terminal alpha peptides of 60 amino acids had maximal β-galactosidase activity in their assay (Nishiyama et al., Protein Sci. 24(5):599-603 (2015)). The following wild type LacZ alpha region from strain MG1655 truncated at residue 60 was used: MTMITDSLAVVLQRRDWENPGVTQLNRLAAHPPFASWRNSEEARTD RPSQQLRSLNGEWR (SEQ ID NO:1).

The terminator sequence was derived from the rrnBT2 terminator described by Orosz et al. (Orosz et al., Eur. J. Biochem. 201(3):653-9 (1991)).

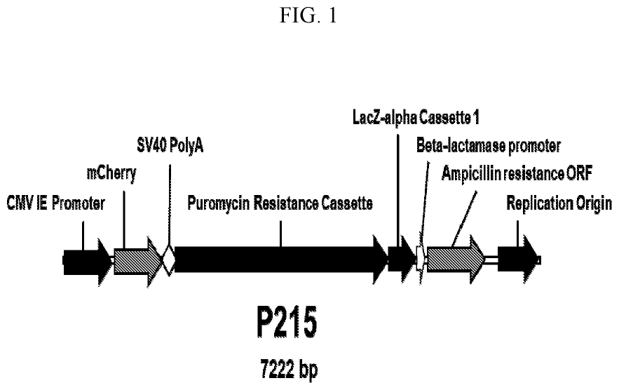

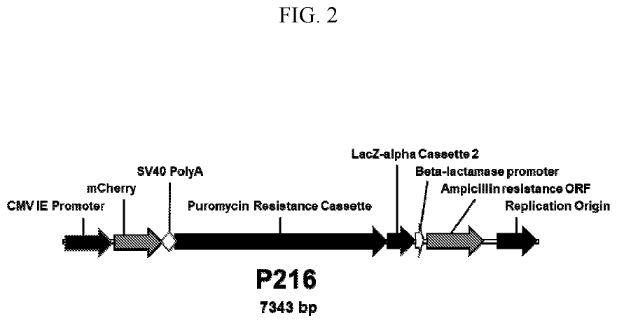

The P215 (SEQ ID NO:9) (FIG. 1) and P216 (SEQ ID NO:10) (FIG. 2) plasmids were constructed by gene synthesis at GeneWiz (South Plainfield, N.J.). The plasmids contain an ampicillin resistance cassette and a 4.9 kb transgene.

### Results

Plasmids without antibiotic selection markers are desirable for gene therapy applications and cell line development for therapeutic products. It has also been reported that plasmid backbones 1 kb or smaller were useful in avoiding gene silencing when delivered to animals in vivo. The purpose of these experiments was to explore a new strategy for developing a small metabolic selection marker for selection of plasmid-containing cells in E. coli.

It was hypothesized that plasmids that express the alpha peptide of β-galactosidase could complement the LacZΔ15 allele in Top10 cells, completing the lactose operon and allowing cells to grow on minimal media with lactose as the sole carbon source.

In Example 1, whether pUC19 and pBluescript vectors that express lacZa fusion peptides could complement TOP10 cells and allow them to grow on minimal media with lactose was tested. These experiments were unsuccessful.

Based on the hypothesis that the lacZa fusion proteins encoded by these vectors were suboptimal at complementing the LacZΔ15 mutation and were not expressed at high enough levels to enable growth on Lactose-containing minimal media, vectors were synthesized with new lacZa expression cassettes. The ability of these vectors to complement the LacZΔ15 mutation was tested. Ten nanograms (ng) of plasmids P215 and P216, and pBluescript II were transformed into 50 μl OneShot Top10 cells. The cells were incubated with DNA on ice for 20 minutes, heat shocked at 42° C. for 30 seconds, and returned to incubate on ice for 1 minute. 450 μl of SOC was added to the cells, and the cells were incubated at 37° C. for 1 hour while shaking. 250 μl of cells were removed and the remaining cells were returned to the incubator. The extracted cells were washed two times with 500 μl of D-PBS and resuspended in 200 μl of D-PBS after the last wash. 50 μl of cells were plated on LB-carbenicillin (100), M9+glucose, and M9+lactose plates, and the plates were incubated at 37° C. After 4.5 hours post heat shock, the remaining cells from the incubator were washed, as described above, and plated onto M9+glucose and M9+lactose plates. The plates were incubated at 37° C. overnight.

Transformations plated on M9+glucose made a lawn of cells, indicating that Top10 host cells can grow on these plates. Transformations plated on LB-carbenicillin (100) produced lots of colonies as well. The LB-carbenicillin plates were stored at 4° C. The M9+lactose plates remained at 37° C. to incubate for 24 more hours.

Transformations allowed to recover for either one hour or for four hours both produced a large number of colonies when plated on the M9+lactose plates. There were no colonies on the pBluescript II transformations confirming the results from Example 1, indicating that pBluescript II was unable to produce enough β-galactosidase through complementation of the LacZΔ15 mutation to allow growth on lactose minimal media. The plates were stored at 4° C.

Natural plasmids such as ColE1 are efficiently maintained in E. coli hosts in the absence of antibiotic selection while the pUC series of vectors can be lost from cells at a high rate in the absence of selection (Summers, Molecular Microbiology 29: 1137-1145 (1998)). However, given the much slower growth rate of P215 and P216-transformed cells on minimal media versus rich LB media, it would be much faster and cheaper for plasmid DNA purification to grow cell cultures in LB in the absence of selection if the frequency of plasmid loss was not too high. β-galactosidase alpha-complementation plasmid-containing cells are easily distinguished from plasmid-free cells grown on LB-IPTG-XGAL plates since the β-galactosidase hydrolyzes the XGAL (5-bromo-4-chloro-3-indolyl-β-D-galactopyranoside) indicator turning the cells blue. This assay was used to investigate the frequency of plasmid loss when these cells are grown in the absence of antibiotics in LB media.

Pure populations of cells were obtained by streaking cells on LB-IPTG-XGAL plates, and colonies that contained plasmids turned blue. Most of the colonies streaked on the plates were blue, as expected.

After obtaining a pure population of cells, serial cultures of the cells were grown. A single blue colony was picked and grown in 2 mls of LB media in a 15 ml tube. The culture was incubated overnight at 37° C. while shaking.

Cells from the cultures were streaked onto LB-IPTG-XGAL plates, and the plates were incubated overnight at 37° C. Colonies on the re-streaked plates were blue. A single colony was inoculated in 50 mls of LB in a 250 ml flask and incubated overnight at 37° C. while shaking.

50 μl of a 10⁻⁴ dilution of the overnight cultures were plated onto LB-IPTG-XGAL plates. The plates were incubated overnight at 37° C. 1 μl of the 50 ml cultures was diluted to a new culture of 50 mls of LB (50,000-fold dilution). The cultures were grown overnight at 37° C.

After incubation overnight, all colonies on the plate were observed to be blue. 50 μl of a 10⁻⁴ dilution of the 50 ml culture from the previous night were plated on LB-IPTG-XGAL plate. 1 μl of the 50 ml cultures from the previous night was diluted to a new culture of 50 mls of LB (50,000-fold dilution). The cultures were grown overnight at 37° C.

After incubation overnight, there were about 1000 colonies observed on the plates with 50 μl of the 10⁻⁴ dilution. All of the colonies of the P215 transformation were blue, and there were only 3 white colonies observed on the P216 transformation plate. The results indicated that plasmids P215 and P216 were stable even in the absence of selection. These plasmids are 7.2 and 7.3 kb for P215 and P216, respectively. From a single colony to 50 mls and then diluted 1:50,000 and grown to confluence twice suggests that the cells could be grown to a volume of 1.25×10⁸ liters without selection while still retaining the plasmid in most of the cells. The transformation efficiency was similar when cells were allowed to recover for one hour versus four hours in SOC media post-heat shock.

The alpha complementation plasmids constructed complemented the LacZΔ15 mutation in Top10 cells allowing growth on minimal media with lactose as the sole carbon source. These plasmids were also found to be stable in LB liquid cultures in the absence of selective pressure.

### Example 3: Reducing the Size of β-Galactosidase-α Complementation Plasmids

In previous experiments, expression of the β-galactosidase alpha peptide from the P215 and P216 plasmids was demonstrated to be useful as selection marker on plasmids, replacing antibiotic resistance genes. Next it was sought to define which regions of the plasmids were essential for plasmid selection and replication in E. coli with the goal of defining the smallest possible replicon.

### Results

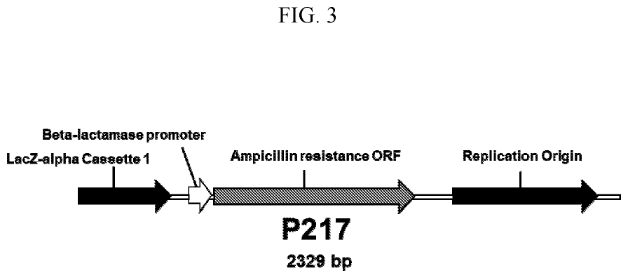

Using standard cloning techniques, the mCherry and puromycin resistance genes were removed from plasmid P215 to create plasmid P217 (SEQ ID NO:11) (FIG. 3).

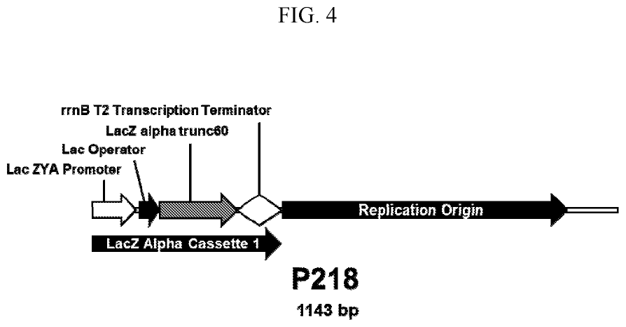

From plasmid P217, standard cloning techniques were used to remove the ampicillin resistance gene. Ligated DNA was transformed into 50 μl of TOP10 cells, incubated on ice for 20 minutes, heat shocked for 30 seconds, and incubated on ice for an additional 3 minutes. After incubation, 450 μl of SOC media was added to the cells, and the cells were incubated at 37° C. for 1 hour while shaking. The cells were pelleted and washed 3 times with 1 ml of d-PBS. Cells were plated onto M9-lactose plates and incubated at 37° C. for two days. Colonies from the transformation were picked and streaked onto an LB-IPTG-XGAL plate. The resulting colonies were blue for each clone. A single clone was picked (Clone P218 (SEQ ID NO:12; FIG. 4)), and DNA sequencing confirmed that the desired deletion had been created.

To further decrease the size of the β-galactosidase selection cassette, the rrnBT2 transcription terminator (SEQ ID NO:7) was deleted. In addition to the possibility that this sequence was not necessary to maintain transcript stability, it was reported that read-through transcription from promoters upstream of the pUC57/pMB1 origin can increase copy number by increasing transcription through the replication primer region of the origin (Panayotatos, Nucleic Acid Res. 12(6):2641-8 (1984); Oka et al., Mol Gen Genet. 172(2):151-9 (1979)).

Using standard cloning techniques, colonies were obtained for the deletion construct P219 (SEQ ID NO:13; FIG. 5). The deletion was confirmed through DNA sequencing.

The minimal β-galactosidase expression cassette/replication origin cassette that was elucidated by this work (SEQ ID NO:18) is 938 bp. It fulfills the goal of being smaller than 1 kb in order to avoid DNA silencing in mammalian cells associated with larger plasmid backbones (Lu et al., Mol. Ther. 20(11):2111-9 (2012))).

### Example 4: Creation of β-Galactosidase-α Complementation Vector with Firefly Luciferase Expression Cassette

In the examples provided above, plasmids that use alpha complementation of a β-galactosidase mutation as a selection marker instead of an antibiotic resistance gene were constructed. To determine whether DNA replication was still efficient when the plasmid size increases, the minimal β-galactosidase expression cassette/replication origin sequence defined above (SEQ ID NO:18) was used to replace the antibiotic selection marker and replication origin of an existing plasmid using standard cloning techniques.

The CMV promoter-luciferase-polyA expression cassette from the GWIZ-Luciferase plasmid (SEQ ID NO:16) was cloned into P219 using standard cloning techniques. Transformation into One Shot TOP10 cells, plating onto M9+Lactose plates, and incubation for 2 days at 37° C. produced large colonies. Colonies were re-streaked onto LB-IPTG-XGAL plates and incubated overnight at 37° C.

Blue colonies of the transformation reaction were screened for inserts using primers CNFOR (SEQ ID NO:14); and P455R2 (SEQ ID NO:15). Two PCR-positive colonies were picked and used to inoculate a 6 ml LB culture, which was grown at 37° C. DNA was isolated from the cultures and the DNA yields were estimated by measuring their OD₂₆₀ with a Spectrophotometer (Table 1).

TABLE 1
DNA yields for selected clones

| | A260 |
|---|---|
| Sample | Concentration |
| name | (ng/ul) |
| P469-1 | 132.69 |
| P469-2 | 506.91 |

200 mls of LB in a 500 ml flask was inoculated with a single blue colony for clone P469-2 and grown for 18 hours at 37° C. in a shaker incubator. DNA was purified from this culture using a Qiagen HiSpeed MaxiPrep kit and 440 μg of DNA was recovered. Plasmid P469-2 (SEQ ID NO:17) was sequenced confirmed at GeneWiz.

In this example, the kanamycin resistance gene and replication origin of GWIZ-Luciferase was successfully replaced by the minimal β-galactosidase/replication origin defined above. An acceptable plasmid yield was achieved when this clone was grown without selective pressure in LB media.

### Example 5: Testing β-Galactosidase-α Complementation Vector Function in Various E. coli Strains

To identify additional E. coli strains where the β-galactosidase alpha peptide can be used as a selectable marker instead of an antibiotic resistance gene, one of the plasmids constructed above was tested by DNA transfection into 8 different strains.

TABLE 2
Bacterial Strains

| Strain | Vendor | Genotype |
|---|---|---|
| Top10 | Thermo-Fisher | F- mcrA Δ(mrr-hsdRMS-mcrBC) Φ80lacZΔM15 Δ lacX74 |
| | | recA1 araD139 Δ(araleu)7697 galU galK rpsL (StrR) |
| | | endA1 nupG |
| NEB 5- | New England | fhuA2 Δ(argF-lacZ)U169 phoA glnV44 Φ80 Δ(lacZ)M15 |
| alpha | Biolabs | gyrA96 recA1 relA1 endA1 thi-1 hsdR17 |
| GT115 | InVivogen | F- mcrA Δ(mrr-hsdRMS-mcrBC) φ80lacZΔM15 ΔlacX74 |
| | | recA1 rspL (StrA) endA1 Δdcm uidA(ΔMluI)::pir-116 |
| | | ΔsbcC-sbcD |
| NEB Stable | New England | F′ proA+B+ lacI^{q} Δ(lacZ)M15 zzf::Tn10 (Tet^{R}) Δ(ara-leu) |
| | Biolabs | 7697 araD139 fhuA ΔlacX74 galK16 galE15 e14- |
| | | Φ80dlacZΔM15 recA1 relA1 endA1 nupG rpsL (Str^{R}) rph |
| | | spoT1 Δ(mrr-hsdRMS-mcrBC) |
| Stellar | Takara Bio USA | F-, endA1, supE44, thi-1, recA1, relA1, gyrA96, phoA, |
| | | Φ80d lacZΔ M15, Δ(lacZYA-argF) U169, Δ(mrr-hsdRMS- |
| | | mcrBC), ΔmcrA, λ- |
| DH10B | Thermo-Fisher | F- mcrA Δ(mrr-hsdRMS-mcrBC) φ80lacZΔM15 ΔlacX74 |
| | | recA1 endA1 araD139 Δ (ara, leu)7697 galU galK λ- rpsL |
| | | nupG/pMON14272/pMON7124 |
| Stbl3 | Thermo-Fisher | F⁻mcrB mrrhsdS20(r_{B}⁻, m_{B}⁻) recA13 supE44 ara-14 galK2 |
| | | lacY1 proA2 rpsL20(Str^{R}) xyl-5 λ⁻leumtl-1 |
| XL1-blue | Agilent | recA1 endA1 gyrA96 thi-1 hsdR17 supE44 relA1 lac [F′ |
| | | proAB lacI^{q}ZΔM15 Tn10 (Tetr)]. |

### Results

50 μl of the E. coli strains in Table 2 were incubated with 1 ng of plasmid P469-2 on ice in a sterile microfuge tube for 30 minutes. The cells were heat shocked for 30 seconds at 42° C. and incubated on ice for 1 minute. 450 μl SOC media was added to all cells except NEB-Stable cells. 450 μl of NEB-Stable outgrowth medium (supplied by the manufacturer) was added to the transformed NEB-Stable cells. The cells were incubated at 37° C. for 1 hour while shaking. The cells were pelleted and washed 3 times with 1 ml of D-PBS. Cells were plated onto M9-lactose plates and incubated at 37° C. for three days.

As expected, no colonies were detected on plates from the Stbl3-transformed cells that were included as a negative control. Five of the strains (Top10, GT115, NEB-Stable, Stellar, and DH10B) had normal-sized colonies. Two strains (NEB-Alpha and XL1-Blue) had small colonies. This was expected since a similar strain to NEB-alpha (DH5alpha) and XL1-Blue contain a mutation in the purB gene that results in slow growth on minimal media (Jung et al. Appl Environ. Micro. 76: 6307-6309 (2010)).

XL1-blue and NEB-Alpha plates were incubated for an additional day at 37° C. Pure colonies were obtained by streaking colonies from the M9-lactose plates onto LB-IPTG-XGAL plates and incubating at 37° C. Blue colonies (plasmid containing cells) were streaked a second time onto an LB-IPTG-XGAL plate and incubated at 37° C. which produced mostly blue cells.

All of the tested strains that contained the Φ80dlacZΔM415 marker could be transformed by the β-galactosidase alpha peptide expression plasmid P469-2 and selected on M9 minimal media with lactose as the sole carbon source. Plasmid P469-2 transfectants of strain XL1-blue that contains the marker lacl^{q}ZΔM15 on the F episome were also selectable on M9-Lactose plates. Hence, seven commercially available E. coli strains have been demonstrated to be compatible with the β-galactosidase selectable marker.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

SEQUENCE LISTING

| | |
|---|---|
| <110> | Janssen Biotech, Inc. |
| <120> | Beta-Galactosidase Alpha Peptide As A Non-Antibiotic Selection |
| | Marker and Uses Thereof |
| <130> | 688097-553U5 |
| <160> | 20 |
| <170> | PatentIn version 3.5 |
| <210> | 1 |
| <211> | 60 |
| <212> | PRT |
| <213> | Artificial Sequence |
| <220> | |
| <223> | Truncated LazC alpha peptide |
| <400> | 1 |

```
Met Thr Met Ile Thr Asp Ser Leu Ala Val Val Leu Gln Arg Arg Asp
1 5 10 15
Trp Glu Asn Pro Gly Val Thr Gln Leu Asn Arg Leu Ala Ala His Pro
                        20 25 30
Pro Phe Ala Ser Trp Arg Asn Ser Glu Glu Ala Arg Thr Asp Arg Pro
                35 40 45
Ser Gln Gln Leu Arg Ser Leu Asn Gly Glu Trp Arg
        50 55 60
```

| | |
|---|---|
| <210> | 2 |
| <211> | 419 |
| <212> | DNA |
| <213> | Artificial Sequence |
| <220> | |
| <223> | LacZ alpha cassette 1 |
| <400> | 2 |

| | |
|---|---|
| agcgggcagt gagcgcaacg caattaatgt gagttagctc actcattagg caccccaggc | 60 |
| tttacacttt atgcttccgg ctcgtatgtt gtgtggaatt gtgagcggat aacaatttca | 120 |
| cacaggaaac agctatgacc atgattacgg attcactggc cgtcgtttta caacgtcgtg | 180 |
| actgggaaaa ccctggcgtt acccaactta atcgccttgc agcacatccc cctttcgcca | 240 |
| gctggcgtaa tagcgaagag gcccgcaccg atcgcccttc ccaacagttg cgcagcctga | 300 |
| atggcgaatg gcgctgaggc ccggagggtg gcgggcagga cgcccgccat aaactgccag | 360 |
| gcatcaaatt aagcagaagg ccatcctgac ggatggcctt tttgcgtttc tacaaactc | 419 |

| | |
|---|---|
| <210> | 3 |
| <211> | 540 |
| <212> | DNA |
| <213> | Artificial Sequence |
| <220> | |
| <223> | LacZ alpha cassette 2 |
| <400> | 3 |

| | |
|---|---|
| cacgtctcta tggaaatatg acggtgttca caaagttcct taaattttac ttttggttac | 60 |
| atattttttc tttttgaaac caaatcttta tctttgtagc actttcacgg tagcgaaacg | 120 |
| ttagtttgaa tggaaagatg cctgcagaca cataaagaca ccaaactctc atcaatagtt | 180 |
| ccgtaaattt ttattgacag aacttattga cggcagtggc aggtgtcata aaaaaaacca | 240 |
| tgagggtaat aaataatgac catgattacg gattcactgg ccgtcgtttt acaacgtcgt | 300 |
| gactgggaaa accctggcgt tacccaactt aatcgccttg cagcacatcc ccctttcgcc | 360 |
| agctggcgta atagcgaaga ggcccgcacc gatcgccctt cccaacagtt gcgcagcctg | 420 |
| aatggcgaat ggcgctgagg cccggagggt ggcgggcagg acgcccgcca taaactgcca | 480 |
| ggcatcaaat taagcagaag gccatcctga cggatggcct ttttgcgttt ctacaaactc | 540 |

| | |
|---|---|
| <210> | 4 |
| <211> | 96 |
| <212> | DNA |
| <213> | Artificial Sequence |
| <220> | |
| <223> | LacZYA promoter |
| <400> | 4 |

| | |
|---|---|
| agcgggcagt gagcgcaacg caattaatgt gagttagctc actcattagg caccccaggc | 60 |
| tttacacttt atgcttccgg ctcgtatgtt gtgtgg | 96 |

| | |
|---|---|
| <210> | 5 |
| <211> | 38 |
| <212> | DNA |
| <213> | Artificial Sequence |
| <220> | |
| <223> | Lac Operator |
| <400> | 5 |

| | |
|---|---|
| aattgtgagc ggataacaat ttcacacagg aaacagct | 38 |

| | |
|---|---|
| <210> | 6 |
| <211> | 183 |
| <212> | DNA |
| <213> | Artificial Sequence |
| <220> | |
| <223> | Truncated LacZ alpha peptide nucleotide sequence |
| <400> | 6 |

| | |
|---|---|
| atgaccatga ttacggattc actggccgtc gttttacaac gtcgtgactg ggaaaaccct | 60 |
| ggcgttaccc aacttaatcg ccttgcagca catccccctt tcgccagctg gcgtaatagc | 120 |
| gaagaggccc gcaccgatcg cccttcccaa cagttgcgca gcctgaatgg cgaatggcgc | 180 |
| tga | 183 |

| | |
|---|---|
| <210> | 7 |
| <211> | 102 |
| <212> | DNA |
| <213> | Artificial Sequence |
| <220> | |
| <223> | rrnBT2 transcription terminator |
| <400> | 7 |

| | |
|---|---|
| ggcccggagg gtggcgggca ggacgcccgc cataaactgc caggcatcaa attaagcaga | 60 |
| aggccatcct gacggatggc ctttttgcgt ttctacaaac tc | 102 |

| | |
|---|---|
| <210> | 8 |
| <211> | 255 |
| <212> | DNA |
| <213> | Artificial Sequence |
| <220> | |
| <223> | OmpF promoter |
| <400> | 8 |

| | |
|---|---|
| cacgtctcta tggaaatatg acggtgttca caaagttcct taaattttac ttttggttac | 60 |
| atattttttc tttttgaaac caaatcttta tctttgtagc actttcacgg tagcgaaacg | 120 |
| ttagtttgaa tggaaagatg cctgcagaca cataaagaca ccaaactctc atcaatagtt | 180 |
| ccgtaaattt ttattgacag aacttattga cggcagtggc aggtgtcata aaaaaaacca | 240 |
| tgagggtaat aaata | 255 |

| | |
|---|---|
| <210> | 9 |
| <211> | 7222 |
| <212> | DNA |
| <213> | Artificial Sequence |
| <220> | |
| <223> | P215 |
| <400> | 9 |

| | |
|---|---|
| taactataac ggtcctaagg tagcgaagct cttcagatgg acagtcagac tgaagagcct | 60 |
| ctcttaaggt agctcgagga gcttggccca ttgcatacgt tgtatccata tcataatatg | 120 |
| tacatttata ttggctcatg tccaacatta ccgccatgtt gacattgatt attgactagt | 180 |
| tattaatagt aatcaattac ggggtcatta gttcatagcc catatatgga gttccgcgtt | 240 |
| acataactta cggtaaatgg cccgcctggc tgaccgccca acgacccccg cccattgacg | 300 |
| tcaataatga cgtatgttcc catagtaacg ccaataggga ctttccattg acgtcaatgg | 360 |
| gtggagtatt tacggtaaac tgcccacttg gcagtacatc aagtgtatca tatgccaagt | 420 |
| acgcccccta ttgacgtcaa tgacggtaaa tggcccgcct ggcattatgc ccagtacatg | 480 |
| accttatggg actttcctac ttggcagtac atctacgtat tagtcatcgc tattaccatg | 540 |
| gtgatgcggt tttggcagta catcaatggg cgtggatagc ggtttgactc acggggattt | 600 |
| ccaagtctcc accccattga cgtcaatggg agtttgtttt ggcaccaaaa tcaacgggac | 660 |
| tttccaaaat gtcgtaacaa ctccgcccca ttgacgcaaa tgggcggtag gcgtgtacgg | 720 |
| tgggaggtct atataagcag agctcgttta gtgaaccgtc ggcgcgccgc caccatggtg | 780 |
| agcaagggcg aggaggataa catggccatc atcaaggagt tcatgcgctt caaggtgcac | 840 |
| atggagggct ccgtgaacgg ccacgagttc gagatcgagg gcgagggcga gggccgcccc | 900 |
| tacgagggca cccagaccgc caagctgaag gtgaccaagg gtggccccct gcccttcgcc | 960 |
| tgggacatcc tgtcccctca gttcatgtac ggctccaagg cctacgtgaa gcaccccgcc | 1020 |
| gacatccccg actacttgaa gctgtccttc cccgagggct tcaagtggga gcgcgtgatg | 1080 |
| aacttcgagg acggcggcgt ggtgaccgtg acccaggact cctccctgca ggacggcgag | 1140 |
| ttcatctaca aggtgaagct gcgcggcacc aacttcccct ccgacggccc cgtaatgcag | 1200 |
| aagaagacca tgggctggga ggcctcctcc gagcggatgt accccgagga cggcgccctg | 1260 |
| aagggcgaga tcaagcagag gctgaagctg aaggacggcg gccactacga cgctgaggtc | 1320 |
| aagaccacct acaaggccaa gaagcccgtg cagctgcccg gcgcctacaa cgtcaacatc | 1380 |
| aagttggaca tcacctccca caacgaggac tacaccatcg tggaacagta cgaacgcgcc | 1440 |
| gagggccgcc actccaccgg cggcatggac gagctgtaca agtagtctag agatacattg | 1500 |
| atgagtttgg acaaaccaca actagaatgc agtgaaaaaa atgctttatt tgtgaaattt | 1560 |
| gtgatgctat tgctttattt gtaaccatta taagctgcaa taaacaagtt aacaacaaca | 1620 |
| attgcattca ttttatgttt caggttcagg gggaggtgtg ggaggttttt taaagcaagt | 1680 |
| aaaacctcta caaatgtggt atggctgatt atgatcgcgg ccgcgttcca tgtccttata | 1740 |
| tggactcatc tttgcctatt gcgacacaca ctcagtgaac acctactacg cgctgcaaag | 1800 |
| agccccgcag gcctgaggtg cccccacctc accactcttc ctatttttgt gtaaaaatcc | 1860 |
| agcttcttgt caccacctcc aaggaggggg aggaggagga aggcaggttc ctctaggctg | 1920 |
| agccgaatgc ccctctgtgg tcccacgcca ctgatcgctg catgcccacc acctgggtac | 1980 |
| acacagtctg tgattcccgg agcagaacgg accctgccca cccggtcttg tgtgctactc | 2040 |
| agtggacaga cccaaggcaa gaaagggtga caaggacagg gtcttcccag gctggctttg | 2100 |
| agttcctagc accgccccgc ccccaatcct ctgtggcaca tggagtcttg gtccccagag | 2160 |
| tcccccagcg gcctccagat ggtctgggag ggcagttcag ctgtggctgc gcatagcaga | 2220 |
| catacaacgg acggtgggcc cagacccagg ctgtgtagac ccagcccccc cgccccgcag | 2280 |
| tgcctaggtc acccactaac gccccaggcc ttgtcttggc tgggcgtgac tgttaccctc | 2340 |
| aaaagcaggc agctccaggg taaaaggtgc cctgccctgt agagcccacc ttccttccca | 2400 |
| gggctgcggc tgggtaggtt tgtagccttc atcacgggcc acctccagcc actggaccgc | 2460 |
| tggcccctgc cctgtcctgg ggagtgtggt cctgcgactt ctaagtggcc gcaagccacc | 2520 |
| tgactccccc aacaccacac tctacctctc aagcccaggt ctctccctag tgacccaccc | 2580 |
| agcacattta gctagctgag ccccacagcc agaggtcctc aggccctgct ttcagggcag | 2640 |
| ttgctctgaa gtcggcaagg gggagtgact gcctggccac tccatgccct ccaagagctt | 2700 |
| cttctgcagg agcgtacaga acccagggcc ctggcacccg tgcagaccct ggcccacccc | 2760 |
| acctgggcgc tcagtgccca agagatgtcc acacctagga tgtcccgcgg tgggtggggg | 2820 |
| gcccgagaga cgggcaggcc gggggcaggc ctggccatgc ggggccgaac cgggcactgc | 2880 |
| ccagcgtggg gcgcgggggc cacggcgcgc gcccccagcc cccgggccca gcaccccaag | 2940 |
| gcggccaacg ccaaaactct ccctcctcct cttcctcaat ctcgctctcg ctcttttttt | 3000 |
| ttttcgcaaa aggaggggag agggggtaaa aaaatgctgc actgtgcggc gaagccggtg | 3060 |
| agtgagcggc gcggggccaa tcagcgtgcg ccgttccgaa agttgccttt tatggctcga | 3120 |
| gtggccgcgg cggcgcccta taaaacccag cggcgcgacg cgccaccacc gccgagaccg | 3180 |
| cgtccgcccc gcgagcacag agcctcgcct ttgccgatcc gccgcccgtc cacacccgcc | 3240 |
| gccaggtaag cccggccagc cgaccggggc aggcggctca cggcccggcc gcaggaggcc | 3300 |
| gcggcccctt cgcccgtgca gagccgccgt ctgggccgca gcggggggcg catggggggg | 3360 |
| gaaccggacc gccgtggggg gcgcgggaga agcccctggg cctccggaga tgggggacac | 3420 |
| cccacgccag ttcggaggcg cgaggccgcg ctcgggaggc gcgctccggg ggtgccgctc | 3480 |
| tcggggcggg ggcaaccggc ggggtctttg tctgagccgg gctcttgcca atggggatcg | 3540 |
| cagggtgggc gcggcggagc ccccgccagg cccggtgggg gctggggcgc cattgcgcgt | 3600 |
| gcgcgctggt cctttgggcg ctaactgcgt gcgcgctggg aattggcgct aattgcgcgt | 3660 |
| gcgcgctggg actcaaggcg ctaactgcgc gtgcgttctg gggcccgggg tgccgcggcc | 3720 |
| tgggctgggg cgaaggcggg ctcggccgga aggggtgggg tcgccgcggc tcccgggcgc | 3780 |
| ttgcgcgcac ttcctgcccg agccgctggc cgcccgaggg tgtggccgct gcgtgcgcgc | 3840 |
| gcgccgaccc ggcgctgttt gaaccgggcg gaggcggggc tggcgcccgg ttgggagggg | 3900 |
| gttggggcct ggcttcctgc cgcgcgccgc ggggacgcct ccgaccagtg tttgcctttt | 3960 |
| atggtaataa cgcggccggc ccggcttcct ttgtccccaa tctgggcgcg cgccggcgcc | 4020 |
| ccctggcggc ctaaggactc ggctcgccgg aagtggccag ggcgggggcg acctcggctc | 4080 |
| acagcgcgcc cggctattct cgcagctcgc caccatgacc gagtacaagc ccacggtgcg | 4140 |
| cctcgccacc cgcgacgacg tcccccgggc cgtacgcacc ctcgccgccg cgttcgccga | 4200 |
| ctaccccgcc acgcgccaca ccgttgaccc ggaccgccac atcgagcggg tcaccgagct | 4260 |
| gcaagaactc ttcctcacgc gcgtcgggct cgacatcggc aaggtgtggg tcgcggacga | 4320 |
| cggcgccgcg gtggcggtct ggaccacgcc ggagagcgtc gaagcggggg cggtgttcgc | 4380 |
| cgagatcggc ccgcgcatgg ccgagttgag cggttcccgg ctggccgcgc agcaacagat | 4440 |
| ggaaggcctc ctggcgccgc accggcccaa ggagcccgcg tggttcctgg ccaccgtcgg | 4500 |
| cgtctcgccc gaccaccagg gcaagggtct gggcagcgcc gtcgtgctcc ccggagtgga | 4560 |
| ggcggccgag cgcgccgggg tgcccgcctt cctggagacc tccgcgcccc gcaacctccc | 4620 |
| cttctacgag cggctcggct tcaccgtcac cgccgacgtc gaggtgcccg aaggaccgcg | 4680 |
| cacctggtgc atgacccgca agcccggtgc ctgatgtgcc ttctagttgc cagccatctg | 4740 |
| ttgtttgccc ctcccccgtg ccttccttga ccctggaagg tgccactccc actgtccttt | 4800 |
| cctaataaaa tgaggaaatt gcatcgcatt gtctgagtag gtgtcattct attctggggg | 4860 |
| gtggggtggg gcaggacagc aagggggagg attgggaaga caatagcagg catgctgggg | 4920 |
| atgcggtggg ctctatggta gggataacag ggtaatagcg ggcagtgagc gcaacgcaat | 4980 |
| taatgtgagt tagctcactc attaggcacc ccaggcttta cactttatgc ttccggctcg | 5040 |
| tatgttgtgt ggaattgtga gcggataaca atttcacaca ggaaacagct atgaccatga | 5100 |
| ttacggattc actggccgtc gttttacaac gtcgtgactg ggaaaaccct ggcgttaccc | 5160 |
| aacttaatcg ccttgcagca catccccctt tcgccagctg gcgtaatagc gaagaggccc | 5220 |
| gcaccgatcg cccttcccaa cagttgcgca gcctgaatgg cgaatggcgc tgaggcccgg | 5280 |
| agggtggcgg gcaggacgcc cgccataaac tgccaggcat caaattaagc agaaggccat | 5340 |
| cctgacggat ggcctttttg cgtttctaca aactctggca aacagctatt atgggtatta | 5400 |
| tgggtgacgt caggtggcac ttttcgggga aatgtgcgcg gaacccctat ttgtttattt | 5460 |
| ttctaaatac attcaaatat gtatccgctc atgagacaat aaccctgata aatgcttcaa | 5520 |
| taatattgaa aaaggaagag tatgagtatt caacatttcc gtgtcgccct tattcccttt | 5580 |
| tttgcggcat tttgccttcc tgtttttgct cacccagaaa cgctggtgaa agtaaaagat | 5640 |
| gctgaagatc agttgggtgc acgagtgggt tacatcgaac tggatctcaa cagcggtaag | 5700 |
| atccttgaga gttttcgccc cgaagaacgt tttccaatga tgagcacttt taaagttctg | 5760 |
| ctatgtggcg cggtattatc ccgtattgac gccgggcaag agcaactcgg tcgccgcata | 5820 |
| cactattctc agaatgactt ggttgagtac tcaccagtca cagaaaagca tcttacggat | 5880 |
| ggcatgacag taagagaatt atgcagtgct gccataacca tgagtgataa cactgcggcc | 5940 |
| aacttacttc tgacaacgat cggaggaccg aaggagctaa ccgctttttt gcacaacatg | 6000 |
| ggggatcatg taactcgcct tgatcgttgg gaaccggagc tgaatgaagc cataccaaac | 6060 |
| gacgagcgtg acaccacgat gcctgtagca atggcaacaa cgttgcgcaa actattaact | 6120 |
| ggcgaactac ttactctagc ttcccggcaa caattaatag actggatgga ggcggataaa | 6180 |
| gttgcaggac cacttctgcg ctcggccctt ccggctggct ggtttattgc tgataaatct | 6240 |
| ggagccggtg agcgtgggtc tcgcggtatc attgcagcac tggggccaga tggtaagccc | 6300 |
| tcccgtatcg tagttatcta cacgacgggg agtcaggcaa ctatggatga acgaaataga | 6360 |
| cagatcgctg agataggtgc ctcactgatt aagcattggt aactgtcaga ccaagtttac | 6420 |
| tcatatatac tttagattga tttaaaactt catttttaat ttaaaaggat ctaggtgaag | 6480 |
| atcctttttg ataatctcat gaccaaaatc ccttaacgtg agttttcgtt ccactgagcg | 6540 |
| tcagaccccg tagaaaagat caaaggatct tcttgagatc ctttttttct gcgcgtaatc | 6600 |
| tgctgcttgc aaacaaaaaa accaccgcta ccagcggtgg tttgtttgcc ggatcaagag | 6660 |
| ctaccaactc tttttccgaa ggtaactggc ttcagcagag cgcagatacc aaatactgtt | 6720 |
| cttctagtgt agccgtagtt aggccaccac ttcaagaact ctgtagcacc gcctacatac | 6780 |
| ctcgctctgc taatcctgtt accagtggct gctgccagtg gcgataagtc gtgtcttacc | 6840 |
| gggttggact caagacgata gttaccggat aaggcgcagc ggtcgggctg aacggggggt | 6900 |
| tcgtgcacac agcccagctt ggagcgaacg acctacaccg aactgagata cctacagcgt | 6960 |
| gagctatgag aaagcgccac gcttcccgaa gggagaaagg cggacaggta tccggtaagc | 7020 |
| ggcagggtcg gaacaggaga gcgcacgagg gagcttccag ggggaaacgc ctggtatctt | 7080 |
| tatagtcctg tcgggtttcg ccacctctga cttgagcgtc gatttttgtg atgctcgtca | 7140 |
| ggggggcgga gcctatggaa aaacgccagc aacgcggcct ttttacggtt cctggccttt | 7200 |
| tgctggcctt ttgctcacat gt | 7222 |

| | |
|---|---|
| <210> | 10 |
| <211> | 7343 |
| <212> | DNA |
| <213> | Artificial Sequence |
| <220> | |
| <223> | P216 |
| <400> | 10 |

| | |
|---|---|
| taactataac ggtcctaagg tagcgaagct cttcagatgg acagtcagac tgaagagcct | 60 |
| ctcttaaggt agctcgagga gcttggccca ttgcatacgt tgtatccata tcataatatg | 120 |
| tacatttata ttggctcatg tccaacatta ccgccatgtt gacattgatt attgactagt | 180 |
| tattaatagt aatcaattac ggggtcatta gttcatagcc catatatgga gttccgcgtt | 240 |
| acataactta cggtaaatgg cccgcctggc tgaccgccca acgacccccg cccattgacg | 300 |
| tcaataatga cgtatgttcc catagtaacg ccaataggga ctttccattg acgtcaatgg | 360 |
| gtggagtatt tacggtaaac tgcccacttg gcagtacatc aagtgtatca tatgccaagt | 420 |
| acgcccccta ttgacgtcaa tgacggtaaa tggcccgcct ggcattatgc ccagtacatg | 480 |
| accttatggg actttcctac ttggcagtac atctacgtat tagtcatcgc tattaccatg | 540 |
| gtgatgcggt tttggcagta catcaatggg cgtggatagc ggtttgactc acggggattt | 600 |
| ccaagtctcc accccattga cgtcaatggg agtttgtttt ggcaccaaaa tcaacgggac | 660 |
| tttccaaaat gtcgtaacaa ctccgcccca ttgacgcaaa tgggcggtag gcgtgtacgg | 720 |
| tgggaggtct atataagcag agctcgttta gtgaaccgtc ggcgcgccgc caccatggtg | 780 |
| agcaagggcg aggaggataa catggccatc atcaaggagt tcatgcgctt caaggtgcac | 840 |
| atggagggct ccgtgaacgg ccacgagttc gagatcgagg gcgagggcga gggccgcccc | 900 |
| tacgagggca cccagaccgc caagctgaag gtgaccaagg gtggccccct gcccttcgcc | 960 |
| tgggacatcc tgtcccctca gttcatgtac ggctccaagg cctacgtgaa gcaccccgcc | 1020 |
| gacatccccg actacttgaa gctgtccttc cccgagggct tcaagtggga gcgcgtgatg | 1080 |
| aacttcgagg acggcggcgt ggtgaccgtg acccaggact cctccctgca ggacggcgag | 1140 |
| ttcatctaca aggtgaagct gcgcggcacc aacttcccct ccgacggccc cgtaatgcag | 1200 |
| aagaagacca tgggctggga ggcctcctcc gagcggatgt accccgagga cggcgccctg | 1260 |
| aagggcgaga tcaagcagag gctgaagctg aaggacggcg gccactacga cgctgaggtc | 1320 |
| aagaccacct acaaggccaa gaagcccgtg cagctgcccg gcgcctacaa cgtcaacatc | 1380 |
| aagttggaca tcacctccca caacgaggac tacaccatcg tggaacagta cgaacgcgcc | 1440 |
| gagggccgcc actccaccgg cggcatggac gagctgtaca agtagtctag agatacattg | 1500 |
| atgagtttgg acaaaccaca actagaatgc agtgaaaaaa atgctttatt tgtgaaattt | 1560 |
| gtgatgctat tgctttattt gtaaccatta taagctgcaa taaacaagtt aacaacaaca | 1620 |
| attgcattca ttttatgttt caggttcagg gggaggtgtg ggaggttttt taaagcaagt | 1680 |
| aaaacctcta caaatgtggt atggctgatt atgatcgcgg ccgcgttcca tgtccttata | 1740 |
| tggactcatc tttgcctatt gcgacacaca ctcagtgaac acctactacg cgctgcaaag | 1800 |
| agccccgcag gcctgaggtg cccccacctc accactcttc ctatttttgt gtaaaaatcc | 1860 |
| agcttcttgt caccacctcc aaggaggggg aggaggagga aggcaggttc ctctaggctg | 1920 |
| agccgaatgc ccctctgtgg tcccacgcca ctgatcgctg catgcccacc acctgggtac | 1980 |
| acacagtctg tgattcccgg agcagaacgg accctgccca cccggtcttg tgtgctactc | 2040 |
| agtggacaga cccaaggcaa gaaagggtga caaggacagg gtcttcccag gctggctttg | 2100 |
| agttcctagc accgccccgc ccccaatcct ctgtggcaca tggagtcttg gtccccagag | 2160 |
| tcccccagcg gcctccagat ggtctgggag ggcagttcag ctgtggctgc gcatagcaga | 2220 |
| catacaacgg acggtgggcc cagacccagg ctgtgtagac ccagcccccc cgccccgcag | 2280 |
| tgcctaggtc acccactaac gccccaggcc ttgtcttggc tgggcgtgac tgttaccctc | 2340 |
| aaaagcaggc agctccaggg taaaaggtgc cctgccctgt agagcccacc ttccttccca | 2400 |
| gggctgcggc tgggtaggtt tgtagccttc atcacgggcc acctccagcc actggaccgc | 2460 |
| tggcccctgc cctgtcctgg ggagtgtggt cctgcgactt ctaagtggcc gcaagccacc | 2520 |
| tgactccccc aacaccacac tctacctctc aagcccaggt ctctccctag tgacccaccc | 2580 |
| agcacattta gctagctgag ccccacagcc agaggtcctc aggccctgct ttcagggcag | 2640 |
| ttgctctgaa gtcggcaagg gggagtgact gcctggccac tccatgccct ccaagagctt | 2700 |
| cttctgcagg agcgtacaga acccagggcc ctggcacccg tgcagaccct ggcccacccc | 2760 |
| acctgggcgc tcagtgccca agagatgtcc acacctagga tgtcccgcgg tgggtggggg | 2820 |
| gcccgagaga cgggcaggcc gggggcaggc ctggccatgc ggggccgaac cgggcactgc | 2880 |
| ccagcgtggg gcgcgggggc cacggcgcgc gcccccagcc cccgggccca gcaccccaag | 2940 |
| gcggccaacg ccaaaactct ccctcctcct cttcctcaat ctcgctctcg ctcttttttt | 3000 |
| ttttcgcaaa aggaggggag agggggtaaa aaaatgctgc actgtgcggc gaagccggtg | 3060 |
| agtgagcggc gcggggccaa tcagcgtgcg ccgttccgaa agttgccttt tatggctcga | 3120 |
| gtggccgcgg cggcgcccta taaaacccag cggcgcgacg cgccaccacc gccgagaccg | 3180 |
| cgtccgcccc gcgagcacag agcctcgcct ttgccgatcc gccgcccgtc cacacccgcc | 3240 |
| gccaggtaag cccggccagc cgaccggggc aggcggctca cggcccggcc gcaggaggcc | 3300 |
| gcggcccctt cgcccgtgca gagccgccgt ctgggccgca gcggggggcg catggggggg | 3360 |
| gaaccggacc gccgtggggg gcgcgggaga agcccctggg cctccggaga tgggggacac | 3420 |
| cccacgccag ttcggaggcg cgaggccgcg ctcgggaggc gcgctccggg ggtgccgctc | 3480 |
| tcggggcggg ggcaaccggc ggggtctttg tctgagccgg gctcttgcca atggggatcg | 3540 |
| cagggtgggc gcggcggagc ccccgccagg cccggtgggg gctggggcgc cattgcgcgt | 3600 |
| gcgcgctggt cctttgggcg ctaactgcgt gcgcgctggg aattggcgct aattgcgcgt | 3660 |
| gcgcgctggg actcaaggcg ctaactgcgc gtgcgttctg gggcccgggg tgccgcggcc | 3720 |
| tgggctgggg cgaaggcggg ctcggccgga aggggtgggg tcgccgcggc tcccgggcgc | 3780 |
| ttgcgcgcac ttcctgcccg agccgctggc cgcccgaggg tgtggccgct gcgtgcgcgc | 3840 |
| gcgccgaccc ggcgctgttt gaaccgggcg gaggcggggc tggcgcccgg ttgggagggg | 3900 |
| gttggggcct ggcttcctgc cgcgcgccgc ggggacgcct ccgaccagtg tttgcctttt | 3960 |
| atggtaataa cgcggccggc ccggcttcct ttgtccccaa tctgggcgcg cgccggcgcc | 4020 |
| ccctggcggc ctaaggactc ggctcgccgg aagtggccag ggcgggggcg acctcggctc | 4080 |
| acagcgcgcc cggctattct cgcagctcgc caccatgacc gagtacaagc ccacggtgcg | 4140 |
| cctcgccacc cgcgacgacg tcccccgggc cgtacgcacc ctcgccgccg cgttcgccga | 4200 |
| ctaccccgcc acgcgccaca ccgttgaccc ggaccgccac atcgagcggg tcaccgagct | 4260 |
| gcaagaactc ttcctcacgc gcgtcgggct cgacatcggc aaggtgtggg tcgcggacga | 4320 |
| cggcgccgcg gtggcggtct ggaccacgcc ggagagcgtc gaagcggggg cggtgttcgc | 4380 |
| cgagatcggc ccgcgcatgg ccgagttgag cggttcccgg ctggccgcgc agcaacagat | 4440 |
| ggaaggcctc ctggcgccgc accggcccaa ggagcccgcg tggttcctgg ccaccgtcgg | 4500 |
| cgtctcgccc gaccaccagg gcaagggtct gggcagcgcc gtcgtgctcc ccggagtgga | 4560 |
| ggcggccgag cgcgccgggg tgcccgcctt cctggagacc tccgcgcccc gcaacctccc | 4620 |
| cttctacgag cggctcggct tcaccgtcac cgccgacgtc gaggtgcccg aaggaccgcg | 4680 |
| cacctggtgc atgacccgca agcccggtgc ctgatgtgcc ttctagttgc cagccatctg | 4740 |
| ttgtttgccc ctcccccgtg ccttccttga ccctggaagg tgccactccc actgtccttt | 4800 |
| cctaataaaa tgaggaaatt gcatcgcatt gtctgagtag gtgtcattct attctggggg | 4860 |
| gtggggtggg gcaggacagc aagggggagg attgggaaga caatagcagg catgctgggg | 4920 |
| atgcggtggg ctctatggta gggataacag ggtaatcacg tctctatgga aatatgacgg | 4980 |
| tgttcacaaa gttccttaaa ttttactttt ggttacatat tttttctttt tgaaaccaaa | 5040 |
| tctttatctt tgtagcactt tcacggtagc gaaacgttag tttgaatgga aagatgcctg | 5100 |
| cagacacata aagacaccaa actctcatca atagttccgt aaatttttat tgacagaact | 5160 |
| tattgacggc agtggcaggt gtcataaaaa aaaccatgag ggtaataaat aatgaccatg | 5220 |
| attacggatt cactggccgt cgttttacaa cgtcgtgact gggaaaaccc tggcgttacc | 5280 |
| caacttaatc gccttgcagc acatccccct ttcgccagct ggcgtaatag cgaagaggcc | 5340 |
| cgcaccgatc gcccttccca acagttgcgc agcctgaatg gcgaatggcg ctgaggcccg | 5400 |
| gagggtggcg ggcaggacgc ccgccataaa ctgccaggca tcaaattaag cagaaggcca | 5460 |
| tcctgacgga tggccttttt gcgtttctac aaactctggc aaacagctat tatgggtatt | 5520 |
| atgggtgacg tcaggtggca cttttcgggg aaatgtgcgc ggaaccccta tttgtttatt | 5580 |
| tttctaaata cattcaaata tgtatccgct catgagacaa taaccctgat aaatgcttca | 5640 |
| ataatattga aaaaggaaga gtatgagtat tcaacatttc cgtgtcgccc ttattccctt | 5700 |
| ttttgcggca ttttgccttc ctgtttttgc tcacccagaa acgctggtga aagtaaaaga | 5760 |
| tgctgaagat cagttgggtg cacgagtggg ttacatcgaa ctggatctca acagcggtaa | 5820 |
| gatccttgag agttttcgcc ccgaagaacg ttttccaatg atgagcactt ttaaagttct | 5880 |
| gctatgtggc gcggtattat cccgtattga cgccgggcaa gagcaactcg gtcgccgcat | 5940 |
| acactattct cagaatgact tggttgagta ctcaccagtc acagaaaagc atcttacgga | 6000 |
| tggcatgaca gtaagagaat tatgcagtgc tgccataacc atgagtgata acactgcggc | 6060 |
| caacttactt ctgacaacga tcggaggacc gaaggagcta accgcttttt tgcacaacat | 6120 |
| gggggatcat gtaactcgcc ttgatcgttg ggaaccggag ctgaatgaag ccataccaaa | 6180 |
| cgacgagcgt gacaccacga tgcctgtagc aatggcaaca acgttgcgca aactattaac | 6240 |
| tggcgaacta cttactctag cttcccggca acaattaata gactggatgg aggcggataa | 6300 |
| agttgcagga ccacttctgc gctcggccct tccggctggc tggtttattg ctgataaatc | 6360 |
| tggagccggt gagcgtgggt ctcgcggtat cattgcagca ctggggccag atggtaagcc | 6420 |
| ctcccgtatc gtagttatct acacgacggg gagtcaggca actatggatg aacgaaatag | 6480 |
| acagatcgct gagataggtg cctcactgat taagcattgg taactgtcag accaagttta | 6540 |
| ctcatatata ctttagattg atttaaaact tcatttttaa tttaaaagga tctaggtgaa | 6600 |
| gatccttttt gataatctca tgaccaaaat cccttaacgt gagttttcgt tccactgagc | 6660 |
| gtcagacccc gtagaaaaga tcaaaggatc ttcttgagat cctttttttc tgcgcgtaat | 6720 |
| ctgctgcttg caaacaaaaa aaccaccgct accagcggtg gtttgtttgc cggatcaaga | 6780 |
| gctaccaact ctttttccga aggtaactgg cttcagcaga gcgcagatac caaatactgt | 6840 |
| tcttctagtg tagccgtagt taggccacca cttcaagaac tctgtagcac cgcctacata | 6900 |
| cctcgctctg ctaatcctgt taccagtggc tgctgccagt ggcgataagt cgtgtcttac | 6960 |
| cgggttggac tcaagacgat agttaccgga taaggcgcag cggtcgggct gaacgggggg | 7020 |
| ttcgtgcaca cagcccagct tggagcgaac gacctacacc gaactgagat acctacagcg | 7080 |
| tgagctatga gaaagcgcca cgcttcccga agggagaaag gcggacaggt atccggtaag | 7140 |
| cggcagggtc ggaacaggag agcgcacgag ggagcttcca gggggaaacg cctggtatct | 7200 |
| ttatagtcct gtcgggtttc gccacctctg acttgagcgt cgatttttgt gatgctcgtc | 7260 |
| aggggggcgg agcctatgga aaaacgccag caacgcggcc tttttacggt tcctggcctt | 7320 |
| ttgctggcct tttgctcaca tgt | 7343 |

| | |
|---|---|
| <210> | 11 |
| <211> | 2329 |
| <212> | DNA |
| <213> | Artificial Sequence |
| <220> | |
| <223> | P217 |
| <400> | 11 |

| | |
|---|---|
| agcgggcagt gagcgcaacg caattaatgt gagttagctc actcattagg caccccaggc | 60 |
| tttacacttt atgcttccgg ctcgtatgtt gtgtggaatt gtgagcggat aacaatttca | 120 |
| cacaggaaac agctatgacc atgattacgg attcactggc cgtcgtttta caacgtcgtg | 180 |
| actgggaaaa ccctggcgtt acccaactta atcgccttgc agcacatccc cctttcgcca | 240 |
| gctggcgtaa tagcgaagag gcccgcaccg atcgcccttc ccaacagttg cgcagcctga | 300 |
| atggcgaatg gcgctgaggc ccggagggtg gcgggcagga cgcccgccat aaactgccag | 360 |
| gcatcaaatt aagcagaagg ccatcctgac ggatggcctt tttgcgtttc tacaaactct | 420 |
| ggcaaacagc tattatgggt attatgggtg acgtcaggtg gcacttttcg gggaaatgtg | 480 |
| cgcggaaccc ctatttgttt atttttctaa atacattcaa atatgtatcc gctcatgaga | 540 |
| caataaccct gataaatgct tcaataatat tgaaaaagga agagtatgag tattcaacat | 600 |
| ttccgtgtcg cccttattcc cttttttgcg gcattttgcc ttcctgtttt tgctcaccca | 660 |
| gaaacgctgg tgaaagtaaa agatgctgaa gatcagttgg gtgcacgagt gggttacatc | 720 |
| gaactggatc tcaacagcgg taagatcctt gagagttttc gccccgaaga acgttttcca | 780 |
| atgatgagca cttttaaagt tctgctatgt ggcgcggtat tatcccgtat tgacgccggg | 840 |
| caagagcaac tcggtcgccg catacactat tctcagaatg acttggttga gtactcacca | 900 |
| gtcacagaaa agcatcttac ggatggcatg acagtaagag aattatgcag tgctgccata | 960 |
| accatgagtg ataacactgc ggccaactta cttctgacaa cgatcggagg accgaaggag | 1020 |
| ctaaccgctt ttttgcacaa catgggggat catgtaactc gccttgatcg ttgggaaccg | 1080 |
| gagctgaatg aagccatacc aaacgacgag cgtgacacca cgatgcctgt agcaatggca | 1140 |
| acaacgttgc gcaaactatt aactggcgaa ctacttactc tagcttcccg gcaacaatta | 1200 |
| atagactgga tggaggcgga taaagttgca ggaccacttc tgcgctcggc ccttccggct | 1260 |
| ggctggttta ttgctgataa atctggagcc ggtgagcgtg ggtctcgcgg tatcattgca | 1320 |
| gcactggggc cagatggtaa gccctcccgt atcgtagtta tctacacgac ggggagtcag | 1380 |
| gcaactatgg atgaacgaaa tagacagatc gctgagatag gtgcctcact gattaagcat | 1440 |
| tggtaactgt cagaccaagt ttactcatat atactttaga ttgatttaaa acttcatttt | 1500 |
| taatttaaaa ggatctaggt gaagatcctt tttgataatc tcatgaccaa aatcccttaa | 1560 |
| cgtgagtttt cgttccactg agcgtcagac cccgtagaaa agatcaaagg atcttcttga | 1620 |
| gatccttttt ttctgcgcgt aatctgctgc ttgcaaacaa aaaaaccacc gctaccagcg | 1680 |
| gtggtttgtt tgccggatca agagctacca actctttttc cgaaggtaac tggcttcagc | 1740 |
| agagcgcaga taccaaatac tgttcttcta gtgtagccgt agttaggcca ccacttcaag | 1800 |
| aactctgtag caccgcctac atacctcgct ctgctaatcc tgttaccagt ggctgctgcc | 1860 |
| agtggcgata agtcgtgtct taccgggttg gactcaagac gatagttacc ggataaggcg | 1920 |
| cagcggtcgg gctgaacggg gggttcgtgc acacagccca gcttggagcg aacgacctac | 1980 |
| accgaactga gatacctaca gcgtgagcta tgagaaagcg ccacgcttcc cgaagggaga | 2040 |
| aaggcggaca ggtatccggt aagcggcagg gtcggaacag gagagcgcac gagggagctt | 2100 |
| ccagggggaa acgcctggta tctttatagt cctgtcgggt ttcgccacct ctgacttgag | 2160 |
| cgtcgatttt tgtgatgctc gtcagggggg cggagcctat ggaaaaacgc cagcaacgcg | 2220 |
| gcctttttac ggttcctggc cttttgctgg ccttttgctc acatgttaac tataacggtc | 2280 |
| ctaaggtagc gaagctcggt gggctctatg gtagggataa cagggtaat | 2329 |

| | |
|---|---|
| <210> | 12 |
| <211> | 1143 |
| <212> | DNA |
| <213> | Artificial Sequence |
| <220> | |
| <223> | P218 |
| <400> | 12 |

| | |
|---|---|
| agcgggcagt gagcgcaacg caattaatgt gagttagctc actcattagg caccccaggc | 60 |
| tttacacttt atgcttccgg ctcgtatgtt gtgtggaatt gtgagcggat aacaatttca | 120 |
| cacaggaaac agctatgacc atgattacgg attcactggc cgtcgtttta caacgtcgtg | 180 |
| actgggaaaa ccctggcgtt acccaactta atcgccttgc agcacatccc cctttcgcca | 240 |
| gctggcgtaa tagcgaagag gcccgcaccg atcgcccttc ccaacagttg cgcagcctga | 300 |
| atggcgaatg gcgctgaggc ccggagggtg gcgggcagga cgcccgccat aaactgccag | 360 |
| gcatcaaatt aagcagaagg ccatcctgac ggatggcctt tttgcgtttc tacaaactca | 420 |
| aaggatcttc ttgagatcct ttttttctgc gcgtaatctg ctgcttgcaa acaaaaaaac | 480 |
| caccgctacc agcggtggtt tgtttgccgg atcaagagct accaactctt tttccgaagg | 540 |
| taactggctt cagcagagcg cagataccaa atactgttct tctagtgtag ccgtagttag | 600 |
| gccaccactt caagaactct gtagcaccgc ctacatacct cgctctgcta atcctgttac | 660 |
| cagtggctgc tgccagtggc gataagtcgt gtcttaccgg gttggactca agacgatagt | 720 |
| taccggataa ggcgcagcgg tcgggctgaa cggggggttc gtgcacacag cccagcttgg | 780 |
| agcgaacgac ctacaccgaa ctgagatacc tacagcgtga gctatgagaa agcgccacgc | 840 |
| ttcccgaagg gagaaaggcg gacaggtatc cggtaagcgg cagggtcgga acaggagagc | 900 |
| gcacgaggga gcttccaggg ggaaacgcct ggtatcttta tagtcctgtc gggtttcgcc | 960 |
| acctctgact tgagcgtcga tttttgtgat gctcgtcagg ggggcggagc ctatggaaaa | 1020 |
| acgccagcaa cgcggccttt ttacggttcc tggccttttg ctggcctttt gctcacatgt | 1080 |
| taactataac ggtcctaagg tagcgaagct cggtgggctc tatggtaggg ataacagggt | 1140 |
| aat | 1143 |

| | |
|---|---|
| <210> | 13 |
| <211> | 1047 |
| <212> | DNA |
| <213> | Artificial Sequence |
| <220> | |
| <223> | P219 |
| <400> | 13 |

| | |
|---|---|
| agcgggcagt gagcgcaacg caattaatgt gagttagctc actcattagg caccccaggc | 60 |
| tttacacttt atgcttccgg ctcgtatgtt gtgtggaatt gtgagcggat aacaatttca | 120 |
| cacaggaaac agctatgacc atgattacgg attcactggc cgtcgtttta caacgtcgtg | 180 |
| actgggaaaa ccctggcgtt acccaactta atcgccttgc agcacatccc cctttcgcca | 240 |
| gctggcgtaa tagcgaagag gcccgcaccg atcgcccttc ccaacagttg cgcagcctga | 300 |
| atggcgaatg gcgctgaaag cttaaaggat cttcttgaga tccttttttt ctgcgcgtaa | 360 |
| tctgctgctt gcaaacaaaa aaaccaccgc taccagcggt ggtttgtttg ccggatcaag | 420 |
| agctaccaac tctttttccg aaggtaactg gcttcagcag agcgcagata ccaaatactg | 480 |
| ttcttctagt gtagccgtag ttaggccacc acttcaagaa ctctgtagca ccgcctacat | 540 |
| acctcgctct gctaatcctg ttaccagtgg ctgctgccag tggcgataag tcgtgtctta | 600 |
| ccgggttgga ctcaagacga tagttaccgg ataaggcgca gcggtcgggc tgaacggggg | 660 |
| gttcgtgcac acagcccagc ttggagcgaa cgacctacac cgaactgaga tacctacagc | 720 |
| gtgagctatg agaaagcgcc acgcttcccg aagggagaaa ggcggacagg tatccggtaa | 780 |
| gcggcagggt cggaacagga gagcgcacga gggagcttcc agggggaaac gcctggtatc | 840 |
| tttatagtcc tgtcgggttt cgccacctct gacttgagcg tcgatttttg tgatgctcgt | 900 |
| caggggggcg gagcctatgg aaaaacgcca gcaacgcggc ctttttacgg ttcctggcct | 960 |
| tttgctggcc ttttgctcac atgttaacta taacggtcct aaggtagcga agctcggtgg | 1020 |
| gctctatggt agggataaca gggtaat | 1047 |

| | |
|---|---|
| <210> | 14 |
| <211> | 25 |
| <212> | DNA |
| <213> | Artificial Sequence |
| <220> | |
| <223> | CNFOR |
| <400> | 14 |

| | |
|---|---|
| tgtgtggaat tgtgagcgga taaca | 25 |

| | |
|---|---|
| <210> | 15 |
| <211> | 27 |
| <212> | DNA |
| <213> | Artificial Sequence |
| <220> | |
| <223> | P455R2 |
| <400> | 15 |

| | |
|---|---|
| tggcgttact atgggaacat acgtcat | 27 |

| | |
|---|---|
| <210> | 16 |
| <211> | 6732 |
| <212> | DNA |
| <213> | Artificial Sequence |
| <220> | |
| <223> | GWIZ luciferase |
| <400> | 16 |

| | |
|---|---|
| tcgcgcgttt cggtgatgac ggtgaaaacc tctgacacat gcagctcccg gagacggtca | 60 |
| cagcttgtct gtaagcggat gccgggagca gacaagcccg tcagggcgcg tcagcgggtg | 120 |
| ttggcgggtg tcggggctgg cttaactatg cggcatcaga gcagattgta ctgagagtgc | 180 |
| accatatgcg gtgtgaaata ccgcacagat gcgtaaggag aaaataccgc atcagattgg | 240 |
| ctattggcca ttgcatacgt tgtatccata tcataatatg tacatttata ttggctcatg | 300 |
| tccaacatta ccgccatgtt gacattgatt attgactagt tattaatagt aatcaattac | 360 |
| ggggtcatta gttcatagcc catatatgga gttccgcgtt acataactta cggtaaatgg | 420 |
| cccgcctggc tgaccgccca acgacccccg cccattgacg tcaataatga cgtatgttcc | 480 |
| catagtaacg ccaataggga ctttccattg acgtcaatgg gtggagtatt tacggtaaac | 540 |
| tgcccacttg gcagtacatc aagtgtatca tatgccaagt acgcccccta ttgacgtcaa | 600 |
| tgacggtaaa tggcccgcct ggcattatgc ccagtacatg accttatggg actttcctac | 660 |
| ttggcagtac atctacgtat tagtcatcgc tattaccatg gtgatgcggt tttggcagta | 720 |
| catcaatggg cgtggatagc ggtttgactc acggggattt ccaagtctcc accccattga | 780 |
| cgtcaatggg agtttgtttt ggcaccaaaa tcaacgggac tttccaaaat gtcgtaacaa | 840 |
| ctccgcccca ttgacgcaaa tgggcggtag gcgtgtacgg tgggaggtct atataagcag | 900 |
| agctcgttta gtgaaccgtc agatcgcctg gagacgccat ccacgctgtt ttgacctcca | 960 |
| tagaagacac cgggaccgat ccagcctccg cggccgggaa cggtgcattg gaacgcggat | 1020 |
| tccccgtgcc aagagtgacg taagtaccgc ctatagactc tataggcaca cccctttggc | 1080 |
| tcttatgcat gctatactgt ttttggcttg gggcctatac acccccgctt ccttatgcta | 1140 |
| taggtgatgg tatagcttag cctataggtg tgggttattg accattattg accactcccc | 1200 |
| tattggtgac gatactttcc attactaatc cataacatgg ctctttgcca caactatctc | 1260 |
| tattggctat atgccaatac tctgtccttc agagactgac acggactctg tatttttaca | 1320 |
| ggatggggtc ccatttatta tttacaaatt cacatataca acaacgccgt cccccgtgcc | 1380 |
| cgcagttttt attaaacata gcgtgggatc tccacgcgaa tctcgggtac gtgttccgga | 1440 |
| catgggctct tctccggtag cggcggagct tccacatccg agccctggtc ccatgcctcc | 1500 |
| agcggctcat ggtcgctcgg cagctccttg ctcctaacag tggaggccag acttaggcac | 1560 |
| agcacaatgc ccaccaccac cagtgtgccg cacaaggccg tggcggtagg gtatgtgtct | 1620 |
| gaaaatgagc gtggagattg ggctcgcacg gctgacgcag atggaagact taaggcagcg | 1680 |
| gcagaagaag atgcaggcag ctgagttgtt gtattctgat aagagtcaga ggtaactccc | 1740 |
| gttgcggtgc tgttaacggt ggagggcagt gtagtctgag cagtactcgt tgctgccgcg | 1800 |
| cgcgccacca gacataatag ctgacagact aacagactgt tcctttccat gggtcttttc | 1860 |
| tgcagtcacc gtcgtcgaca cgtgtgatca gatatcgcgg ccgctctagg aagctttcca | 1920 |
| tggaagacgc caaaaacata aagaaaggcc cggcgccatt ctatccgctg gaagatggaa | 1980 |
| ccgctggaga gcaactgcat aaggctatga agagatacgc cctggttcct ggaacaattg | 2040 |
| cttttacaga tgcacatatc gaggtggaca tcacttacgc tgagtacttc gaaatgtccg | 2100 |
| ttcggttggc agaagctatg aaacgatatg ggctgaatac aaatcacaga atcgtcgtat | 2160 |
| gcagtgaaaa ctctcttcaa ttctttatgc cggtgttggg cgcgttattt atcggagttg | 2220 |
| cagttgcgcc cgcgaacgac atttataatg aacgtgaatt gctcaacagt atgggcattt | 2280 |
| cgcagcctac cgtggtgttc gtttccaaaa aggggttgca aaaaattttg aacgtgcaaa | 2340 |
| aaaagctccc aatcatccaa aaaattatta tcatggattc taaaacggat taccagggat | 2400 |
| ttcagtcgat gtacacgttc gtcacatctc atctacctcc cggttttaat gaatacgatt | 2460 |
| ttgtgccaga gtccttcgat agggacaaga caattgcact gatcatgaac tcctctggat | 2520 |
| ctactggtct gcctaaaggt gtcgctctgc ctcatagaac tgcctgcgtg agattctcgc | 2580 |
| atgccagaga tcctattttt ggcaatcaaa tcattccgga tactgcgatt ttaagtgttg | 2640 |
| ttccattcca tcacggtttt ggaatgttta ctacactcgg atatttgata tgtggatttc | 2700 |
| gagtcgtctt aatgtataga tttgaagaag agctgtttct gaggagcctt caggattaca | 2760 |
| agattcaaag tgcgctgctg gtgccaaccc tattctcctt cttcgccaaa agcactctga | 2820 |
| ttgacaaata cgatttatct aatttacacg aaattgcttc tggtggcgct cccctctcta | 2880 |
| aggaagtcgg ggaagcggtt gccaagaggt tccatctgcc aggtatcagg caaggatatg | 2940 |
| ggctcactga gactacatca gctattctga ttacacccga gggggatgat aaaccgggcg | 3000 |
| cggtcggtaa agttgttcca ttttttgaag cgaaggttgt ggatctggat accgggaaaa | 3060 |
| cgctgggcgt taatcaaaga ggcgaactgt gtgtgagagg tcctatgatt atgtccggtt | 3120 |
| atgtaaacaa tccggaagcg accaacgcct tgattgacaa ggatggatgg ctacattctg | 3180 |
| gagacatagc ttactgggac gaagacgaac acttcttcat cgttgaccgc ctgaagtctc | 3240 |
| tgattaagta caaaggctat caggtggctc ccgctgaatt ggaatccatc ttgctccaac | 3300 |
| accccaacat cttcgacgca ggtgtcgcag gtcttcccga cgatgacgcc ggtgaacttc | 3360 |
| ccgccgccgt tgttgttttg gagcacggaa agacgatgac ggaaaaagag atcgtggatt | 3420 |
| acgtcgccag tcaagtaaca accgcgaaaa agttgcgcgg aggagttgtg tttgtggacg | 3480 |
| aagtaccgaa aggtcttacc ggaaaactcg acgcaagaaa aatcagagag atcctcataa | 3540 |
| aggccaagaa gggcggaaag atcgccgtgt aattctagac caggcgcctg gatccagatc | 3600 |
| acttctggct aataaaagat cagagctcta gagatctgtg tgttggtttt ttgtggatct | 3660 |
| gctgtgcctt ctagttgcca gccatctgtt gtttgcccct cccccgtgcc ttccttgacc | 3720 |
| ctggaaggtg ccactcccac tgtcctttcc taataaaatg aggaaattgc atcgcattgt | 3780 |
| ctgagtaggt gtcattctat tctggggggt ggggtggggc aggacagcaa gggggaggat | 3840 |
| tgggaagaca atagcaggca tgctggggat gcggtgggct ctatgggtac ctctctctct | 3900 |
| ctctctctct ctctctctct ctctctctct cggtacctct ctctctctct ctctctctct | 3960 |
| ctctctctct ctctctcggt accaggtgct gaagaattga cccggttcct cctgggccag | 4020 |
| aaagaagcag gcacatcccc ttctctgtga cacaccctgt ccacgcccct ggttcttagt | 4080 |
| tccagcccca ctcataggac actcatagct caggagggct ccgccttcaa tcccacccgc | 4140 |
| taaagtactt ggagcggtct ctccctccct catcagccca ccaaaccaaa cctagcctcc | 4200 |
| aagagtggga agaaattaaa gcaagatagg ctattaagtg cagagggaga gaaaatgcct | 4260 |
| ccaacatgtg aggaagtaat gagagaaatc atagaatttc ttccgcttcc tcgctcactg | 4320 |
| actcgctgcg ctcggtcgtt cggctgcggc gagcggtatc agctcactca aaggcggtaa | 4380 |
| tacggttatc cacagaatca ggggataacg caggaaagaa catgtgagca aaaggccagc | 4440 |
| aaaaggccag gaaccgtaaa aaggccgcgt tgctggcgtt tttccatagg ctccgccccc | 4500 |
| ctgacgagca tcacaaaaat cgacgctcaa gtcagaggtg gcgaaacccg acaggactat | 4560 |
| aaagatacca ggcgtttccc cctggaagct ccctcgtgcg ctctcctgtt ccgaccctgc | 4620 |
| cgcttaccgg atacctgtcc gcctttctcc cttcgggaag cgtggcgctt tctcaatgct | 4680 |
| cacgctgtag gtatctcagt tcggtgtagg tcgttcgctc caagctgggc tgtgtgcacg | 4740 |
| aaccccccgt tcagcccgac cgctgcgcct tatccggtaa ctatcgtctt gagtccaacc | 4800 |
| cggtaagaca cgacttatcg ccactggcag cagccactgg taacaggatt agcagagcga | 4860 |
| ggtatgtagg cggtgctaca gagttcttga agtggtggcc taactacggc tacactagaa | 4920 |
| ggacagtatt tggtatctgc gctctgctga agccagttac cttcggaaaa agagttggta | 4980 |
| gctcttgatc cggcaaacaa accaccgctg gtagcggtgg tttttttgtt tgcaagcagc | 5040 |
| agattacgcg cagaaaaaaa ggatctcaag aagatccttt gatcttttct acggggtctg | 5100 |
| acgctcagtg gaacgaaaac tcacgttaag ggattttggt catgagatta tcaaaaagga | 5160 |
| tcttcaccta gatcctttta aattaaaaat gaagttttaa atcaatctaa agtatatatg | 5220 |
| agtaaacttg gtctgacagt taccaatgct taatcagtga ggcacctatc tcagcgatct | 5280 |
| gtctatttcg ttcatccata gttgcctgac tccggggggg gggggcgctg aggtctgcct | 5340 |
| cgtgaagaag gtgttgctga ctcataccag gcctgaatcg ccccatcatc cagccagaaa | 5400 |
| gtgagggagc cacggttgat gagagctttg ttgtaggtgg accagttggt gattttgaac | 5460 |
| ttttgctttg ccacggaacg gtctgcgttg tcgggaagat gcgtgatctg atccttcaac | 5520 |
| tcagcaaaag ttcgatttat tcaacaaagc cgccgtcccg tcaagtcagc gtaatgctct | 5580 |
| gccagtgtta caaccaatta accaattctg attagaaaaa ctcatcgagc atcaaatgaa | 5640 |
| actgcaattt attcatatca ggattatcaa taccatattt ttgaaaaagc cgtttctgta | 5700 |
| atgaaggaga aaactcaccg aggcagttcc ataggatggc aagatcctgg tatcggtctg | 5760 |
| cgattccgac tcgtccaaca tcaatacaac ctattaattt cccctcgtca aaaataaggt | 5820 |
| tatcaagtga gaaatcacca tgagtgacga ctgaatccgg tgagaatggc aaaagcttat | 5880 |
| gcatttcttt ccagacttgt tcaacaggcc agccattacg ctcgtcatca aaatcactcg | 5940 |
| catcaaccaa accgttattc attcgtgatt gcgcctgagc gagacgaaat acgcgatcgc | 6000 |
| tgttaaaagg acaattacaa acaggaatcg aatgcaaccg gcgcaggaac actgccagcg | 6060 |
| catcaacaat attttcacct gaatcaggat attcttctaa tacctggaat gctgttttcc | 6120 |
| cggggatcgc agtggtgagt aaccatgcat catcaggagt acggataaaa tgcttgatgg | 6180 |
| tcggaagagg cataaattcc gtcagccagt ttagtctgac catctcatct gtaacatcat | 6240 |
| tggcaacgct acctttgcca tgtttcagaa acaactctgg cgcatcgggc ttcccataca | 6300 |
| atcgatagat tgtcgcacct gattgcccga cattatcgcg agcccattta tacccatata | 6360 |
| aatcagcatc catgttggaa tttaatcgcg gcctcgagca agacgtttcc cgttgaatat | 6420 |
| ggctcataac accccttgta ttactgttta tgtaagcaga cagttttatt gttcatgatg | 6480 |
| atatattttt atcttgtgca atgtaacatc agagattttg agacacaacg tggctttccc | 6540 |
| ccccccccca ttattgaagc atttatcagg gttattgtct catgagcgga tacatatttg | 6600 |
| aatgtattta gaaaaataaa caaatagggg ttccgcgcac atttccccga aaagtgccac | 6660 |
| ctgacgtcta agaaaccatt attatcatga cattaaccta taaaaatagg cgtatcacga | 6720 |
| ggccctttcg tc | 6732 |

| | |
|---|---|
| <210> | 17 |
| <211> | 5070 |
| <212> | DNA |
| <213> | Artificial Sequence |
| <220> | |
| <223> | P469-2 |
| <400> | 17 |

| | |
|---|---|
| tagggataac agggtaatag cgggcagtga gcgcaacgca attaatgtga gttagctcac | 60 |
| tcattaggca ccccaggctt tacactttat gcttccggct cgtatgttgt gtggaattgt | 120 |
| gagcggataa caatttcaca caggaaacag ctatgaccat gattacggat tcactggccg | 180 |
| tcgttttaca acgtcgtgac tgggaaaacc ctggcgttac ccaacttaat cgccttgcag | 240 |
| cacatccccc tttcgccagc tggcgtaata gcgaagaggc ccgcaccgat cgcccttccc | 300 |
| aacagttgcg cagcctgaat ggcgaatggc gctgaaagct taaaggatct tcttgagatc | 360 |
| ctttttttct gcgcgtaatc tgctgcttgc aaacaaaaaa accaccgcta ccagcggtgg | 420 |
| tttgtttgcc ggatcaagag ctaccaactc tttttccgaa ggtaactggc ttcagcagag | 480 |
| cgcagatacc aaatactgtt cttctagtgt agccgtagtt aggccaccac ttcaagaact | 540 |
| ctgtagcacc gcctacatac ctcgctctgc taatcctgtt accagtggct gctgccagtg | 600 |
| gcgataagtc gtgtcttacc gggttggact caagacgata gttaccggat aaggcgcagc | 660 |
| ggtcgggctg aacggggggt tcgtgcacac agcccagctt ggagcgaacg acctacaccg | 720 |
| aactgagata cctacagcgt gagctatgag aaagcgccac gcttcccgaa gggagaaagg | 780 |
| cggacaggta tccggtaagc ggcagggtcg gaacaggaga gcgcacgagg gagcttccag | 840 |
| ggggaaacgc ctggtatctt tatagtcctg tcgggtttcg ccacctctga cttgagcgtc | 900 |
| gatttttgtg atgctcgtca ggggggcgga gcctatggaa aaacgccagc aacgcgggtg | 960 |
| cgcataatgt atattatgtt aaattaacta taacggtcct aaggtagcga atggccattg | 1020 |
| catacgttgt atccatatca taatatgtac atttatattg gctcatgtcc aacattaccg | 1080 |
| ccatgttgac attgattatt gactagttat taatagtaat caattacggg gtcattagtt | 1140 |
| catagcccat atatggagtt ccgcgttaca taacttacgg taaatggccc gcctggctga | 1200 |
| ccgcccaacg acccccgccc attgacgtca ataatgacgt atgttcccat agtaacgcca | 1260 |
| atagggactt tccattgacg tcaatgggtg gagtatttac ggtaaactgc ccacttggca | 1320 |
| gtacatcaag tgtatcatat gccaagtacg ccccctattg acgtcaatga cggtaaatgg | 1380 |
| cccgcctggc attatgccca gtacatgacc ttatgggact ttcctacttg gcagtacatc | 1440 |
| tacgtattag tcatcgctat taccatggtg atgcggtttt ggcagtacat caatgggcgt | 1500 |
| ggatagcggt ttgactcacg gggatttcca agtctccacc ccattgacgt caatgggagt | 1560 |
| ttgttttggc accaaaatca acgggacttt ccaaaatgtc gtaacaactc cgccccattg | 1620 |
| acgcaaatgg gcggtaggcg tgtacggtgg gaggtctata taagcagagc tcgtttagtg | 1680 |
| aaccgtcaga tcgcctggag acgccatcca cgctgttttg acctccatag aagacaccgg | 1740 |
| gaccgatcca gcctccgcgg ccgggaacgg tgcattggaa cgcggattcc ccgtgccaag | 1800 |
| agtgacgtaa gtaccgccta tagactctat aggcacaccc ctttggctct tatgcatgct | 1860 |
| atactgtttt tggcttgggg cctatacacc cccgcttcct tatgctatag gtgatggtat | 1920 |
| agcttagcct ataggtgtgg gttattgacc attattgacc actcccctat tggtgacgat | 1980 |
| actttccatt actaatccat aacatggctc tttgccacaa ctatctctat tggctatatg | 2040 |
| ccaatactct gtccttcaga gactgacacg gactctgtat ttttacagga tggggtccca | 2100 |
| tttattattt acaaattcac atatacaaca acgccgtccc ccgtgcccgc agtttttatt | 2160 |
| aaacatagcg tgggatctcc acgcgaatct cgggtacgtg ttccggacat gggctcttct | 2220 |
| ccggtagcgg cggagcttcc acatccgagc cctggtccca tgcctccagc ggctcatggt | 2280 |
| cgctcggcag ctccttgctc ctaacagtgg aggccagact taggcacagc acaatgccca | 2340 |
| ccaccaccag tgtgccgcac aaggccgtgg cggtagggta tgtgtctgaa aatgagcgtg | 2400 |
| gagattgggc tcgcacggct gacgcagatg gaagacttaa ggcagcggca gaagaagatg | 2460 |
| caggcagctg agttgttgta ttctgataag agtcagaggt aactcccgtt gcggtgctgt | 2520 |
| taacggtgga gggcagtgta gtctgagcag tactcgttgc tgccgcgcgc gccaccagac | 2580 |
| ataatagctg acagactaac agactgttcc tttccatggg tcttttctgc agtcaccgtc | 2640 |
| gtcgacacgt gtgatcagat atcgcggccg ctctaggaag ctttccatgg aagacgccaa | 2700 |
| aaacataaag aaaggcccgg cgccattcta tccgctggaa gatggaaccg ctggagagca | 2760 |
| actgcataag gctatgaaga gatacgccct ggttcctgga acaattgctt ttacagatgc | 2820 |
| acatatcgag gtggacatca cttacgctga gtacttcgaa atgtccgttc ggttggcaga | 2880 |
| agctatgaaa cgatatgggc tgaatacaaa tcacagaatc gtcgtatgca gtgaaaactc | 2940 |
| tcttcaattc tttatgccgg tgttgggcgc gttatttatc ggagttgcag ttgcgcccgc | 3000 |
| gaacgacatt tataatgaac gtgaattgct caacagtatg ggcatttcgc agcctaccgt | 3060 |
| ggtgttcgtt tccaaaaagg ggttgcaaaa aattttgaac gtgcaaaaaa agctcccaat | 3120 |
| catccaaaaa attattatca tggattctaa aacggattac cagggatttc agtcgatgta | 3180 |
| cacgttcgtc acatctcatc tacctcccgg ttttaatgaa tacgattttg tgccagagtc | 3240 |
| cttcgatagg gacaagacaa ttgcactgat catgaactcc tctggatcta ctggtctgcc | 3300 |
| taaaggtgtc gctctgcctc atagaactgc ctgcgtgaga ttctcgcatg ccagagatcc | 3360 |
| tatttttggc aatcaaatca ttccggatac tgcgatttta agtgttgttc cattccatca | 3420 |
| cggttttgga atgtttacta cactcggata tttgatatgt ggatttcgag tcgtcttaat | 3480 |
| gtatagattt gaagaagagc tgtttctgag gagccttcag gattacaaga ttcaaagtgc | 3540 |
| gctgctggtg ccaaccctat tctccttctt cgccaaaagc actctgattg acaaatacga | 3600 |
| tttatctaat ttacacgaaa ttgcttctgg tggcgctccc ctctctaagg aagtcgggga | 3660 |
| agcggttgcc aagaggttcc atctgccagg tatcaggcaa ggatatgggc tcactgagac | 3720 |
| tacatcagct attctgatta cacccgaggg ggatgataaa ccgggcgcgg tcggtaaagt | 3780 |
| tgttccattt tttgaagcga aggttgtgga tctggatacc gggaaaacgc tgggcgttaa | 3840 |
| tcaaagaggc gaactgtgtg tgagaggtcc tatgattatg tccggttatg taaacaatcc | 3900 |
| ggaagcgacc aacgccttga ttgacaagga tggatggcta cattctggag acatagctta | 3960 |
| ctgggacgaa gacgaacact tcttcatcgt tgaccgcctg aagtctctga ttaagtacaa | 4020 |
| aggctatcag gtggctcccg ctgaattgga atccatcttg ctccaacacc ccaacatctt | 4080 |
| cgacgcaggt gtcgcaggtc ttcccgacga tgacgccggt gaacttcccg ccgccgttgt | 4140 |
| tgttttggag cacggaaaga cgatgacgga aaaagagatc gtggattacg tcgccagtca | 4200 |
| agtaacaacc gcgaaaaagt tgcgcggagg agttgtgttt gtggacgaag taccgaaagg | 4260 |
| tcttaccgga aaactcgacg caagaaaaat cagagagatc ctcataaagg ccaagaaggg | 4320 |
| cggaaagatc gccgtgtaat tctagaccag gccctggatc cagatcactt ctggctaata | 4380 |
| aaagatcaga gctctagaga tctgtgtgtt ggttttttgt ggatctgctg tgccttctag | 4440 |
| ttgccagcca tctgttgttt gcccctcccc cgtgccttcc ttgaccctgg aaggtgccac | 4500 |
| tcccactgtc ctttcctaat aaaatgagga aattgcatcg cattgtctga gtaggtgtca | 4560 |
| ttctattctg gggggtgggg tggggcagga cagcaagggg gaggattggg aagacaatag | 4620 |
| caggcatgct ggggatgcgg tgggctctat gggtacctct ctctctctct ctctctctct | 4680 |
| ctctctctct ctctctctgg tacctctctc tctctctctc tctctctctc tctctctctc | 4740 |
| tctggtaccc aggtgctgaa gaattgaccc ggttcctcct gggccagaaa gaagcaggca | 4800 |
| catccccttc tctgtgacac accctgtcca cgcccctggt tcttagttcc agccccactc | 4860 |
| ataggacact catagctcag gagggctccg ccttcaatcc cacccgctaa agtacttgga | 4920 |
| gcggtctctc cctccctcat cagcccacca aaccaaacct agcctccaag agtgggaaga | 4980 |
| aattaaagca agataggcta ttaagtgcag agggagagaa aatgcctcca acatgtgagg | 5040 |
| aagtaatgag agaaatcata gaatttcttc | 5070 |

| | |
|---|---|
| <210> | 18 |
| <211> | 938 |
| <212> | DNA |
| <213> | Artificial Sequence |
| <220> | |
| <223> | Beta-galactosidase expression cassette/pUC57 replication origin |
| <400> | 18 |

| | |
|---|---|
| agcgggcagt gagcgcaacg caattaatgt gagttagctc actcattagg caccccaggc | 60 |
| tttacacttt atgcttccgg ctcgtatgtt gtgtggaatt gtgagcggat aacaatttca | 120 |
| cacaggaaac agctatgacc atgattacgg attcactggc cgtcgtttta caacgtcgtg | 180 |
| actgggaaaa ccctggcgtt acccaactta atcgccttgc agcacatccc cctttcgcca | 240 |
| gctggcgtaa tagcgaagag gcccgcaccg atcgcccttc ccaacagttg cgcagcctga | 300 |
| atggcgaatg gcgctgaaag cttaaaggat cttcttgaga tccttttttt ctgcgcgtaa | 360 |
| tctgctgctt gcaaacaaaa aaaccaccgc taccagcggt ggtttgtttg ccggatcaag | 420 |
| agctaccaac tctttttccg aaggtaactg gcttcagcag agcgcagata ccaaatactg | 480 |
| ttcttctagt gtagccgtag ttaggccacc acttcaagaa ctctgtagca ccgcctacat | 540 |
| acctcgctct gctaatcctg ttaccagtgg ctgctgccag tggcgataag tcgtgtctta | 600 |
| ccgggttgga ctcaagacga tagttaccgg ataaggcgca gcggtcgggc tgaacggggg | 660 |
| gttcgtgcac acagcccagc ttggagcgaa cgacctacac cgaactgaga tacctacagc | 720 |
| gtgagctatg agaaagcgcc acgcttcccg aagggagaaa ggcggacagg tatccggtaa | 780 |
| gcggcagggt cggaacagga gagcgcacga gggagcttcc agggggaaac gcctggtatc | 840 |
| tttatagtcc tgtcgggttt cgccacctct gacttgagcg tcgatttttg tgatgctcgt | 900 |
| caggggggcg gagcctatgg aaaaacgcca gcaacgcg | 938 |

| | |
|---|---|
| <210> | 19 |
| <211> | 615 |
| <212> | DNA |
| <213> | Artificial Sequence |
| <220> | |
| <223> | pUC57 replication origin |
| <400> | 19 |

| | |
|---|---|
| aaaggatctt cttgagatcc tttttttctg cgcgtaatct gctgcttgca aacaaaaaaa | 60 |
| ccaccgctac cagcggtggt ttgtttgccg gatcaagagc taccaactct ttttccgaag | 120 |
| gtaactggct tcagcagagc gcagatacca aatactgttc ttctagtgta gccgtagtta | 180 |
| ggccaccact tcaagaactc tgtagcaccg cctacatacc tcgctctgct aatcctgtta | 240 |
| ccagtggctg ctgccagtgg cgataagtcg tgtcttaccg ggttggactc aagacgatag | 300 |
| ttaccggata aggcgcagcg gtcgggctga acggggggtt cgtgcacaca gcccagcttg | 360 |
| gagcgaacga cctacaccga actgagatac ctacagcgtg agctatgaga aagcgccacg | 420 |
| cttcccgaag ggagaaaggc ggacaggtat ccggtaagcg gcagggtcgg aacaggagag | 480 |
| cgcacgaggg agcttccagg gggaaacgcc tggtatcttt atagtcctgt cgggtttcgc | 540 |
| cacctctgac ttgagcgtcg atttttgtga tgctcgtcag gggggcggag cctatggaaa | 600 |
| aacgccagca acgcg | 615 |

| | |
|---|---|
| <210> | 20 |
| <211> | 237 |
| <212> | DNA |
| <213> | Artificial Sequence |
| <220> | |
| <223> | ColE1 dimer resolution element |
| <400> | 20 |

| | |
|---|---|
| gaaaccatga aaaatggcag cttcagtgga ttaagtgggg gtaatgtggc ctgtaccctc | 60 |
| tggttgcata ggtattcata cggttaaaat ttatcaggcg cgatcgcgca gtttttaggg | 120 |
| tggtttgttg ccatttttac ctgtctgctg ccgtgatcgc gctgaacgcg ttttagcggt | 180 |
| gcgtacaatt aagggattat ggtaaatcca cttactgtct gccctcgtag ccatcga | 237 |

1. A method of using a nucleic acid construct as a selectable marker, the method comprising:
a. contacting a host cell comprising a deletion in a lac operon with the nucleic acid construct, wherein the nucleic acid construct comprises an isolated β-galactosidase expression cassette comprising a nucleic acid sequence encoding the amino-terminal fragment of β-galactosidase operably linked to a promoter; and
b. growing the host cell under conditions wherein the nucleic acid construct is maintained in the host cell.

2. The method of claim 1, wherein the amino-terminal fragment of β-galactosidase comprises an amino acid sequence with at least 75% identity to SEQ ID NO:1.

3. The method of claim 1, wherein the amino-terminal fragment of β-galactosidase comprises an amino acid sequence of SEQ ID NO:1.

4. The method of claim 1, wherein the nucleic acid sequence further comprises a replication origin.

5. The method of claim 4, wherein the replication origin is a high-copy replication origin.

6. The method of claim 5, wherein the high-copy replication origin is the pUC57 replication origin.

7. The method of claim 6, wherein the pUC57 replication origin comprises the nucleic acid sequence of SEQ ID NO:19.

8. The method of claim 1, wherein the isolated β-galactosidase expression cassette further comprises a dimer resolution element.

9. The method of claim 8, wherein the dimer resolution element comprises a nucleic acid sequence comprising a site-specific recombinase recognition site.

10. The method of claim 8, wherein the dimer resolution element further comprises a nucleic acid sequence encoding a site-specific recombinase.

11. The method of claim 8, wherein the host cell comprises a nucleic acid sequence encoding a site-specific recombinase.

12. The method of claim 8, wherein the dimer resolution element is a ColE1 dimer resolution element.

13. The method of claim 12, wherein the ColE1 dimer resolution element comprises the nucleic acid sequence of SEQ ID NO:20.

14. The method of claim 1, wherein the host cell comprises a LacZΔ15 deletion.

15. The method of claim 1, wherein an isolated vector comprises the isolated β-galactosidase expression cassette.

16. The method of claim 15, wherein the isolated vector is less than about 1.5 kilobases in size.

17. The method of claim 15, wherein the isolated vector comprises a nucleic acid sequence selected from the group consisting of SEQ ID NOs:9-13, 17, and 18.

18. A method of generating the isolated vector of claim 15, wherein the method comprises:
a. contacting a host cell with the isolated vector;
b. growing the host cell under conditions to produce the vector;
c. isolating the vector from the host cell.

19. The method of claim 18, wherein the host cell is grown in minimal media.

20. The method of claim 19, wherein the minimal media comprises lactose as the sole carbon source.

21. The method of claim 20, wherein the minimal media comprises about 1% to about 4% weight per volume (w/v) lactose.

22. The method of claim 21, wherein the minimal media comprises about 2% w/v lactose.

23. A kit comprising:
a. an isolated β-galactosidase expression cassette of claim 1; and
b. a host cell comprising a deletion in a lac operon.

24. The kit of claim 23, further comprising minimal media comprising lactose as the sole carbon source.

25. The kit of claim 23, wherein a vector comprises the isolated β-galactosidase expression cassette.

26. The kit of claim 23, wherein the host cell comprises the LacZΔ15 deletion.

27. The kit of claim 26, wherein the host cell is selected from the group consisting of an E. coli host cell and a yeast host cell.


SEQUENCE LISTING (20 sequences)

SEQ ID NO: 1 - protein, 60 aa, Artificial Sequence
  Truncated LazC alpha peptide
MTMITDSLAVVLQRRDWENPGVTQLNRLAAHPPFASWRNSEEARTDRPSQQLRSLNGEWR

SEQ ID NO: 2 - DNA, 419 bp, Artificial Sequence
  LacZ alpha cassette 1
agcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaggcccggagggtggcgggcaggacgcccgccataaactgccaggcatcaaattaagcagaaggccatcctgacggatggcctttttgcgtttctacaaactc

SEQ ID NO: 3 - DNA, 540 bp, Artificial Sequence
  LacZ alpha cassette 2
cacgtctctatggaaatatgacggtgttcacaaagttccttaaattttacttttggttacatattttttctttttgaaaccaaatctttatctttgtagcactttcacggtagcgaaacgttagtttgaatggaaagatgcctgcagacacataaagacaccaaactctcatcaatagttccgtaaatttttattgacagaacttattgacggcagtggcaggtgtcataaaaaaaaccatgagggtaataaataatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaggcccggagggtggcgggcaggacgcccgccataaactgccaggcatcaaattaagcagaaggccatcctgacggatggcctttttgcgtttctacaaactc

SEQ ID NO: 4 - DNA, 96 bp, Artificial Sequence
  LacZYA promoter
agcgggcagtgagcgcaacgcaattaatgtgagttagctcactcattaggcaccccaggctttacactttatgcttccggctcgtatgttgtgtgg

SEQ ID NO: 5 - DNA, 38 bp, Artificial Sequence
  Lac Operator
aattgtgagcggataacaatttcacacaggaaacagct

SEQ ID NO: 6 - DNA, 183 bp, Artificial Sequence
  Truncated LacZ alpha peptide nucleotide sequence
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctga

SEQ ID NO: 7 - DNA, 102 bp, Artificial Sequence
  rrnBT2 transcription terminator
ggcccggagggtggcgggcaggacgcccgccataaactgccaggcatcaaattaagcagaaggccatcctgacggatggcctttttgcgtttctacaaactc

SEQ ID NO: 8 - DNA, 255 bp, Artificial Sequence
  OmpF promoter
cacgtctctatggaaatatgacggtgttcacaaagttccttaaattttacttttggttacatattttttctttttgaaaccaaatctttatctttgtagcactttcacggtagcgaaacgttagtttgaatggaaagatgcctgcagacacataaagacaccaaactctcatcaatagttccgtaaatttttattgacagaacttattgacggcagtggcaggtgtcataaaaaaaaccatgagggtaataaata

SEQ ID NO: 9 - DNA, 7222 bp, Artificial Sequence
  P215
taactataacggtcctaaggtagcgaagctcttcagatggacagtcagactgaagagcctctcttaaggtagctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcggcgcgccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagtctagagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgcggccgcgttccatgtccttatatggactcatctttgcctattgcgacacacactcagtgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggccttgtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagcttcttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagtggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggaggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggctcgccggaagtggccagggcgggggcgacctcggctcacagcgcgcccggctattctcgcagctcgccaccatgaccgagtacaagcccacggtgcgcctcgccacccgcgacgacgtcccccgggccgtacgcaccctcgccgccgcgttcgccgactaccccgccacgcgccacaccgttgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtagggataacagggtaatagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaggcccggagggtggcgggcaggacgcccgccataaactgccaggcatcaaattaagcagaaggccatcctgacggatggcctttttgcgtttctacaaactctggcaaacagctattatgggtattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 10 - DNA, 7343 bp, Artificial Sequence
  P216
taactataacggtcctaaggtagcgaagctcttcagatggacagtcagactgaagagcctctcttaaggtagctcgaggagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcggcgcgccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagtctagagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgcggccgcgttccatgtccttatatggactcatctttgcctattgcgacacacactcagtgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggccttgtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagcttcttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagtggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggaggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggctcgccggaagtggccagggcgggggcgacctcggctcacagcgcgcccggctattctcgcagctcgccaccatgaccgagtacaagcccacggtgcgcctcgccacccgcgacgacgtcccccgggccgtacgcaccctcgccgccgcgttcgccgactaccccgccacgcgccacaccgttgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtagggataacagggtaatcacgtctctatggaaatatgacggtgttcacaaagttccttaaattttacttttggttacatattttttctttttgaaaccaaatctttatctttgtagcactttcacggtagcgaaacgttagtttgaatggaaagatgcctgcagacacataaagacaccaaactctcatcaatagttccgtaaatttttattgacagaacttattgacggcagtggcaggtgtcataaaaaaaaccatgagggtaataaataatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaggcccggagggtggcgggcaggacgcccgccataaactgccaggcatcaaattaagcagaaggccatcctgacggatggcctttttgcgtttctacaaactctggcaaacagctattatgggtattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 11 - DNA, 2329 bp, Artificial Sequence
  P217
agcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaggcccggagggtggcgggcaggacgcccgccataaactgccaggcatcaaattaagcagaaggccatcctgacggatggcctttttgcgtttctacaaactctggcaaacagctattatgggtattatgggtgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttaactataacggtcctaaggtagcgaagctcggtgggctctatggtagggataacagggtaat

SEQ ID NO: 12 - DNA, 1143 bp, Artificial Sequence
  P218
agcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaggcccggagggtggcgggcaggacgcccgccataaactgccaggcatcaaattaagcagaaggccatcctgacggatggcctttttgcgtttctacaaactcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttaactataacggtcctaaggtagcgaagctcggtgggctctatggtagggataacagggtaat

SEQ ID NO: 13 - DNA, 1047 bp, Artificial Sequence
  P219
agcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaaagcttaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttaactataacggtcctaaggtagcgaagctcggtgggctctatggtagggataacagggtaat

SEQ ID NO: 14 - DNA, 25 bp, Artificial Sequence
  CNFOR
tgtgtggaattgtgagcggataaca

SEQ ID NO: 15 - DNA, 27 bp, Artificial Sequence
  P455R2
tggcgttactatgggaacatacgtcat

SEQ ID NO: 16 - DNA, 6732 bp, Artificial Sequence
  GWIZ luciferase
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctaggaagctttccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccaggcgcctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 17 - DNA, 5070 bp, Artificial Sequence
  P469-2
tagggataacagggtaatagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaaagcttaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcgggtgcgcataatgtatattatgttaaattaactataacggtcctaaggtagcgaatggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgcggccgctctaggaagctttccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccaggccctggatccagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacctctctctctctctctctctctctctctctctctctctctctggtacctctctctctctctctctctctctctctctctctctctctggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatttcttc

SEQ ID NO: 18 - DNA, 938 bp, Artificial Sequence
  Beta-galactosidase expression cassette/pUC57 replication origin
agcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctgaaagcttaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcg

SEQ ID NO: 19 - DNA, 615 bp, Artificial Sequence
  pUC57 replication origin
aaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcg

SEQ ID NO: 20 - DNA, 237 bp, Artificial Sequence
  ColE1 dimer resolution element
gaaaccatgaaaaatggcagcttcagtggattaagtgggggtaatgtggcctgtaccctctggttgcataggtattcatacggttaaaatttatcaggcgcgatcgcgcagtttttagggtggtttgttgccatttttacctgtctgctgccgtgatcgcgctgaacgcgttttagcggtgcgtacaattaagggattatggtaaatccacttactgtctgccctcgtagccatcga